(19) United States
(12) Patent Application Publication
(10) Pub. No.: US 2018/0369451 A1
(43) Pub. Date: Dec. 27, 2018

(54) ROTATING DEVICE AND METHOD FOR CONTINUOUS FABRICATION OF LONG BIOLOGICAL TUBES

(71) Applicant: Organovo, Inc., San Diego, CA (US)

(72) Inventors: Harry Scott RAPOPORT, San Diego, CA (US); Emily BOZEK, San Diego, CA (US); Jamie BRUGNANO, Oceansice, CA (US); Vaidehi S. JOSHI, San Diego, CA (US); Steve PENTONEY, Chino Hills, CA (US); Gina SCOTT, San Diego, CA (US)

(73) Assignee: Organovo, Inc., San Diego, CA (US)

(21) Appl. No.: 15/777,443

(22) PCT Filed: Nov. 18, 2016
(86) PCT No.: PCT/US2016/062946

§ 371 Date: May 18, 2018

Related U.S. Application Data
(60) Provisional application No. 62/258,282, filed on Nov. 20, 2015.

(51) Int. Cl. A61L 27/38; C09D 11/14; C12N 5/00; C09D 11/04; B29C 47/08; B29C 47/00; B29C 47/02

(52) U.S. Cl. CPC A61L 27/3808; C09D 11/14; C12N 5/0068; C09D 11/04; B29C 47/0806; B29L 2031/7532; B29C 47/02; B29C 47/0866; B29C 47/0026; B29K 2995/0068; B29K 2995/0065; B29C 47/0004

(57) ABSTRACT

Disclosed are bioprinters for rapidly fabricating biological tubes comprising extruding a bio-ink filament onto a rotating mandrel. Also disclosed are methods of rapidly fabricating biological tubes comprising extruding a bio-ink filament onto a rotating mandrel, maturing the deposited bio-ink filament, and removing the biological tube from the mandrel. Also disclosed are methods of fabricating a multilayered biological tube. Also disclosed are engineered biological tubes prepared using the disclosed methods.

### BACKGROUND OF THE INVENTION

### Field of the Invention

The invention is in the field of tissue engineering. Disclosed are bioprinters for rapidly fabricating biological tubes comprising extruding a bio-ink filament onto a rotating mandrel. Also disclosed are methods of rapidly fabricating biological tubes comprising extruding a bio-ink filament onto a rotating mandrel, maturing the deposited bio-ink filament, and removing the biological tube from the mandrel. Also disclosed are methods of fabricating a multilayered biological tube. Also disclosed are engineered biological tubes prepared using the disclosed methods.

### Background Art

Tissue engineering and regenerative medicine is a field with great promise from both a therapeutic and a research standpoint. Engineered tissues are at the center of many different avenues of tissue engineering research. Methods that can improve the fabrication and formation of these tissues, that can also improve their function both in vitro and in vivo, are needed to facilitate the advancement of this field.

Many human tissues have a tubular geometry including, for example, fluid conduits such as vascular and lymphatic vessels, alimentary tissues such as esophagus, intestine, and pancreatic duct, respiratory tissues such as trachea and bronchiole, reproductive tissues such as fallopian tube and vas deferens, and renal system tissues such as ureter and urethra. Synthetic replacements made from Teflon or Gore-Tex have been used; however, engineered biological tubes composed of viable tissue represent an improvement due to potentially increased compliance, reduced thrombogenicity, and resistance to infections. Engineered tissue potentially also possess the ability to heal, remodel, contract/relax, and secrete tissue products.

The design and engineering of living tissue using bioprinting technologies allows the formation of both reconstituted living tissues for use in product testing and functional organs designed for implantation and therapeutic use. The fabrication of small diameter tubular constructs or cellular cylinders and their use for vascular, pulmonary, and other organ reconstruction requires integrity, uniformity, and mechanical strength of the individual cylinders.

Current methodologies of engineering tubular biological structures do not allow for high-throughput generation of extra-long tubes which will be required for clinical and commercial diagnostic use. Existing methodologies are time consuming, resource intensive, and the large number of critical steps increases the likelihood of technical errors. For example, current methods for engineering tubular biological structures require 35-60 minutes and yield one tubular construct.

Described herein are devices and methods that allow the high-throughput production of longer, more uniform, and optionally multilayered cellular tubes for use as vascular, pulmonary, and other organ-associated tubular structures including multiple tube architectures such as branched tubes.

### SUMMARY OF THE INVENTION

Described herein is a rotating device and method for the fabrication of long tubular biological constructs. The device and method allows for high-throughput formation of tubular structures, which are optionally multilayered. Moreover, the device and method optionally allows the high-throughput formation of extra-long (e.g., >30 cm) tubular structures. The method uses continuous deposition printing or capillary deposition to coil extra-long solid or semi-solid cellular filaments into a tubular structure with an open lumen, which is, for example, 500 μm to 2 cm in diameter.

The technique utilizes a rotating mandrel as a printing surface and simultaneous translation of the printer head down the rotating axis of the mandrel leading to a tightly wrapped coil of cellular material. The solid or semi-solid cellular coiled structure fuses into a tube with an open lumen after, for example, an 8-12 hour incubation. In some embodiments, the method allows the printing of extra-long filaments every 3.5 minutes as compared to previous methods which require 35-60 minutes per filament.

The present invention provides a bioprinter for fabricating biological tubes comprising:

(a) a printer head comprising: a reservoir containing bio-ink and a deposition orifice, the bio-ink a solid or semi-solid continuous filament comprising living cells;

(b) a calibration element for determining the position of the deposition orifice;

(c) an extrusion element for extruding the bio-ink through the deposition orifice by application of pressure;

(d) a rotating mandrel for receiving the extruded bio-ink, the rotating mandrel removable from the device;

(e) a motor for rotating the mandrel; and

(f) a programmable computer processor communicatively connected to the calibration element, the extrusion element, and the motor, the programmable computer processor for regulating motion of the printer head, regulating extrusion of the bio-ink, and regulating the rotation of the mandrel to fabricate a biological tube.

In some embodiments, the motor for rotating the mandrel rotates at 10 rpm to 29 rpm. In some embodiments, the motor for rotating the mandrel rotates at 17 rpm.

In some embodiments, the extrusion element extrudes the bio-ink at a rate of 0.020 mm/s to 0.050 mm/s.

In some embodiments, the extrusion element extrudes the bio-ink at a volume of 0.10 μl/s to 0.50 μl/s.

In some embodiments, the bioprinter further comprises a positioning element for positioning the printer head relative to the mandrel and translating the position of the printer head across the length of the mandrel.

In some embodiments, the positioning element translates the position of the printer head at a rate of 0.03 mm/s to 0.25 mm/s.

In some embodiments, the mandrel is porous. In some embodiments, the mandrel is non-porous.

In some embodiments, the mandrel is permeable to gas, liquid, or both gas and liquid.

In some embodiments, the mandrel is hollow. In some embodiments, the mandrel is solid.

In some embodiments, the mandrel comprises metal, ceramic, polymer, or a combination thereof.

In some embodiments, the mandrel is partially or completely covered with a removable sheath.

In some embodiments, the sheath partially or completely covering the mandrel comprises a polymer.

In some embodiments, the sheath partially or completely covering the mandrel is porous.

In some embodiments, the sheath partially or completely covering the mandrel is permeable to gas, liquid, or both gas and liquid.

In some embodiments, the sheath partially or completely covering the mandrel is non-degradable and has a wall thickness of 50 μm to 1 mm.

In some embodiments, the sheath partially or completely covering the mandrel is degradable and has a wall thickness of 50 μm to 5 mm.

In some embodiments, the bio-ink is a continuous filament more than 50 mm to 1000 mm in length.

In some embodiments, the bio-ink and the deposition orifice are 100 μm to 1000 μm in diameter.

In some embodiments, the bio-ink comprises 50 million cells/mL to 400 million cells/mL. In some embodiments, the bio-ink comprises 200 million cells/mL to 400 million cells/mL. In some embodiments, the bio-ink comprises about 300 million cells/mL.

In some embodiments, the bio-ink consists essentially of cells. In some embodiments, the bio-ink consists of cells, extracellular matrix components, and cellular products secreted by the cells.

In some embodiments, the calibration element comprises an optical detector.

In some embodiments, the bioprinter further comprises a collet to reversibly connect the mandrel to the motor.

In some embodiments, the bioprinter further comprises a deposition needle connecting the reservoir with the deposition orifice.

In some embodiments, the outer diameter of the mandrel is between about 0.1 mm and about 200 mm. In some embodiments, the outer diameter of the mandrel is between about 1 mm and 120 mm.

In some embodiments, the surface velocity of the mandrel relative to the deposition orifice on the printer head is between about 0.05 mm/sec and about 300 mm/sec. In some embodiments, the surface velocity of the mandrel relative to the deposition orifice on the printer head is between about 0.5 mm/sec and about 180 mm/sec.

The present invention provides a method of fabricating a biological tube, the method comprising:

(a) depositing a continuous bio-ink filament, the bio-ink filament solid or semi-solid, the bio-ink filament comprising living cells, onto a rotating biocompatible mandrel; and

(b) maturing the deposited bio-ink filament while on the mandrel in a cell culture media to allow the cells to cohere to form the biological tube.

In some embodiments, the method further comprises:

(c) removing the biological tube from the mandrel; and

(d) maturing the biological tube in a cell culture media.

In some embodiments, the rotating mandrel rotates at 10 rpm to 29 rpm. In some embodiments, the rotating mandrel rotates at 17 rpm.

In some embodiments, the bio-ink is deposited by a bioprinter.

In some embodiments, the bioprinter continuously deposits the bio-ink to fabricate a biological tube at a rate of at least 5 mm/min. In some embodiments, the bioprinter continuously deposits the bio-ink to fabricate a biological tube at a rate of at least 6 mm/min. In some embodiments, the bioprinter continuously deposits the bio-ink to fabricate a biological tube at a rate of at least 7 mm/min. In some embodiments, the bioprinter continuously deposits the bio-ink to fabricate a biological tube at a rate of about 8.6 mm/min.

In some embodiments, the bioprinter continuously deposits the bio-ink at a rate of 0.020 mm/s to 0.050 mm/s. In some embodiments, the bioprinter continuously deposits the bio-ink at a volume of 0.10 μl/s to 0.50 μl/s.

In some embodiments, the bioprinter translates the position of the printer head across the length of the rotating mandrel during deposition of the bio-ink. In some embodiments, the bioprinter translates the position of the printer head at a rate of 0.03 mm/s to 0.25 mm/s.

In some embodiments, the bio-ink is a continuous filament more than 50 mm length. In some embodiments, the bio-ink is a continuous filament more than 100 mm length. In some embodiments, the bio-ink is a continuous filament 100 μm to 1000 μm in diameter.

In some embodiments, the bio-ink comprises 50 million cells/mL to 400 million cells/mL. In some embodiments, the bio-ink comprises 200 million cells/mL to 400 million cells/mL. In some embodiments, the bio-ink comprises about 300 million cells/mL.

In some embodiments, the bio-ink consists essentially of cells. In some embodiments, the bio-ink consists of cells, extracellular matrix components, and cellular products secreted by the cells.

In some embodiments, maturing the deposited bio-ink filament comprises maintaining the filament in culture for 4 hours to 12 hours. In some embodiments, maturing the biological tube comprises maintaining the tube in culture for 8 hours to 2 weeks.

In some embodiments, the biological tube has a uniform outer surface. In some embodiments, the biological tube has a uniform inner surface. In some embodiments, the biological tube has a uniform wall thickness.

In some embodiments, the biological tube has a lumen 250 μm to 10 cm in diameter.

In some embodiments, the biological tube is a multilayered tube and the method further comprises preparing a second continuous bio-ink filament and depositing the second bio-ink filament onto the first deposited bio-ink filament prior to maturing the bio-ink to form the biological tube.

In some embodiments, the multilayered tube is an engineered multilayered vascular tube and at least one of the first and second bio-ink filament comprises living vascular cells.

In some embodiments, the engineered multilayered vascular tube has a lumen 250 μm to 25 mm in diameter.

In some embodiments, the multilayered tube is an engineered multilayered pulmonary tube and at least one of the first and second bio-ink filament comprises living pulmonary cells.

In some embodiments, the engineered multilayered pulmonary tube has a lumen 250 μm to 30 mm in diameter.

In some embodiments, the multilayered tube is an engineered multilayered intestinal tube and at least one of the first and second bio-ink filament comprises living intestinal cells.

In some embodiments, the engineered multilayered intestinal tube has a lumen 2 cm to 5 cm in diameter. In some embodiments, the engineered multilayered intestinal tube has a lumen 8 cm to 12 cm in diameter.

In some embodiments, the biological tube lacks at least one of innervation, lymphatic tissue, perfusable supporting vasculature, and red blood cells.

In some embodiments, the biological tube lacks a synthetic or biological scaffold.

In some embodiments, the method of fabricating a biological tube further comprises depositing a layer of cells onto the bio-ink filament prior to maturing the bio-ink to form the biological tube. In some embodiments, the layer of cells is deposited by a bioprinter. In some embodiments, the bioprinter is an ink-jet bioprinter.

In some embodiments, the method of fabricating a biological tube further comprises depositing a layer of cells onto the mandrel prior to depositing the bio-ink filament onto the mandrel. In some embodiments, the layer of cells is deposited by a bioprinter. In some embodiments, the bioprinter is an ink-jetbioprinter.

The present invention provides a system comprising a permeable, biocompatible tubular sheath and a continuous bio-ink filament comprising living cells wound around the sheath to form a biological tube.

In some embodiments, the system further comprises a mandrel inside the tubular sheath.

In some embodiments, the cells are cohered to form the tube.

The present invention provides a method of fabricating a multilayered biological tube, the method comprising:

(a) depositing a first continuous bio-ink filament, the first bio-ink filament a solid or semi-solid composition comprising living cells onto a rotating biocompatible mandrel;

(b) depositing a second continuous bio-ink filament, the second bio-ink filament a solid or semi-solid composition comprising living cells onto a rotating biocompatible mandrel;

(c) depositing a layer of cells onto the deposited second bio-ink filament; and

(d) maturing the deposited bio-ink filaments and cells while on the mandrel in a cell culture media to allow the cells to cohere to form the biological tube.

In some embodiment, the method further comprises:

(e) removing the biological tube from the mandrel; and

(f) maturing the biological tube in a cell culture media.

In some embodiments, the bio-ink filaments are deposited by a bioprinter and wound around the rotating mandrel.

In some embodiments, the layer of cells is deposited by spraying with a bioprinter.

The present invention provides a method of fabricating a multilayered biological tube, the method comprising:

(a) depositing a layer of cells onto a rotating biocompatible mandrel;

(b) depositing a first continuous bio-ink filament, the first bio-ink filament a solid or semi-solid composition comprising living cells onto a rotating biocompatible mandrel;

(c) depositing a second continuous bio-ink filament, the second bio-ink filament a solid or semi-solid composition comprising living cells onto a rotating biocompatible mandrel; and

(d) maturing the deposited bio-ink filaments and cells while on the mandrel in a cell culture media to allow the cells to cohere to form the biological tube.

In some embodiments, the method further comprises:

(e) removing the biological tube from the mandrel; and

(f) maturing the biological tube in a cell culture media.

In some embodiments, the layer of cells is deposited by spraying with a bioprinter.

In some embodiments, the bio-ink filaments are deposited by a bioprinter and wound around the rotating mandrel.

The present invention provides an engineered biological tube, the tube fabricated by a process comprising:

(a) depositing a continuous bio-ink filament, the bio-ink filament solid or semi-solid, the bio-ink filament comprising living cells onto a rotating biocompatible mandrel; and

(b) maturing the deposited bio-ink filament while on the mandrel in a cell culture media to allow the cells to cohere to form the biological tube.

In some embodiments, the method further comprises

(c) removing the biological tube from the mandrel; and

(d) maturing the biological tube in a cell culture media.

In some embodiments, the mandrel is rotated at 10 rpm to 29 rpm. In some embodiments, the mandrel is rotated at 17 rpm.

In some embodiments, the bio-ink filament is deposited by a bioprinter.

In some embodiments, the bioprinter continuously deposits the bio-ink filament to fabricate a biological tube at a rate of at least 5 mm/min. In some embodiments, the bioprinter continuously deposits the bio-ink filament to fabricate a biological tube at a rate of at least 6 mm/min. In some embodiments, the bioprinter continuously deposits the bio-ink filament to fabricate a biological tube at a rate of at least 7 mm/min. In some embodiments, the bioprinter continuously deposits the bio-ink filament to fabricate a biological tube at a rate of about 8.6 mm/min.

In some embodiments, the bioprinter continuously deposits the bio-ink filament at a rate of 0.020 mm/s to 0.050 mm/s. In some embodiments, the bioprinter continuously deposits the bio-ink filament at a volume of 0.10 μl/s to 0.50 μl/s.

In some embodiments, the bioprinter translates the position of the printer head across the length of the rotating mandrel during deposition of the bio-ink filament.

In some embodiments, the bioprinter translates the position of the printer head at a rate of 0.03 mm/s to 0.25 mm/s.

In some embodiments, the bio-ink is a continuous filament more than 50 mm length. In some embodiments, the bio-ink is a continuous filament more than 100 mm length. In some embodiments, the bio-ink is a continuous filament 100 μm to 1000 μm in diameter.

In some embodiments, the bio-ink comprises 50 million cells/mL to 400 million cells/mL. In some embodiments, the bio-ink comprises 200 million cells/mL to 400 million cells/mL. In some embodiments, the bio-ink comprises about 300 million cells/mL.

In some embodiments, the bio-ink consists essentially of cells. In some embodiments, the bio-ink consists of cells, extracellular matrix components, and cellular products secreted by the cells.

In some embodiments, maturing the deposited bio-ink filament comprises maintaining the filament in culture for 4 hours to 12 hours. In some embodiments, maturing the biological tube comprises maintaining the tube in culture for 8 hours to 2 weeks.

In some embodiments, the biological tube has a uniform outer surface. In some embodiments, the biological tube has a uniform inner surface.

In some embodiments, the biological tube has a uniform wall thickness.

In some embodiments, the biological tube has a lumen 250 μm to 10 cm in diameter.

In some embodiments, the biological tube is a multilayered tube and the process further comprises preparing a second continuous bio-ink filament and depositing the second bio-ink filament onto the first deposited bio-ink filament prior to maturing the bio-ink to form the biological tube.

In some embodiments, the multilayered tube is an engineered multilayered vascular tube and at least one of the first and second bio-ink filament comprises living vascular cells.

In some embodiments, the engineered multilayered vascular tube has a lumen 250 μm to 25 mm in diameter.

In some embodiments, the multilayered tube is an engineered multilayered pulmonary tube and at least one of the first and second bio-ink filament comprises living pulmonary cells.

In some embodiments, the engineered multilayered pulmonary tube has a lumen 250 μm to 30 mm in diameter.

In some embodiments, the multilayered tube is an engineered multilayered intestinal tube and the bio-ink filament comprising living intestinal cells.

In some embodiments, the engineered multilayered intestinal tube has a lumen 2 cm to 5 cm in diameter. In some embodiments, the engineered multilayered intestinal tube has a lumen 8 cm to 12 cm in diameter.

In some embodiments, the biological tube lacks at least one of innervation, lymphatic tissue, perfusable supporting vasculature, and red blood cells.

In some embodiments, the biological tube lacks a synthetic or biological scaffold.

In some embodiments, the process further comprises depositing a layer of cells onto the bio-ink filament prior to maturing the bio-ink to form the biological tube. In some embodiments, the layer of cells is deposited by a bioprinter. In some embodiments, the bioprinter is an ink-jet bioprinter.

In some embodiments, the process further comprises depositing a layer of cells onto the mandrel prior to depositing the bio-ink filament onto the mandrel. In some embodiments, the layer of cells is deposited by a bioprinter. In some embodiments, the bioprinter is an ink-jet bioprinter.

### BRIEF DESCRIPTION OF THE DRAWINGS

The accompanying drawings, which are incorporated herein and form a part of the specification, illustrate one or more embodiments of the present invention and, together with the description, further serve to explain the principles of the invention and to enable a person skilled in the pertinent art to make and use the invention. The following drawings are given by way of illustration only, and thus are not intended to limit the scope of the present invention.

FIG. 1 is a photograph of the bioprinter apparatus described herein; in this case, a bioprinter apparatus including a motor attached to a collet holding a mandrel, onto which a printer head deposits a continuous bio-ink filament through a deposition needle with an orifice.

FIG. 2A is a photograph of the biological tube described in Example 2 prepared using a 50:50 mixture of normal human lung fibroblasts (NHLFs) and bronchial smooth muscle cells (BSMCs) in NOVOGEL® 3.0 immediately after fabrication.

FIG. 2B is a photograph of the biological tube described in Example 2 on day 1 after fabrication.

FIG. 2C is a photograph of a histological section of the biological tube described in Example 2 stained using the hematoxylin & eosin (H&E) stain on day 1 after fabrication.

FIG. 3A is a photograph of the biological tube described in Example 3 prepared using a 50:50 mixture of NHLFs and BSMCs in collagen hydrogel immediately after fabrication.

FIG. 3B is a photograph of the biological tube described in Example 3 on day 1 after fabrication.

FIG. 3C is a photograph of a histological section of the biological tube described in Example 2 stained using the H&E stain on day 1 after fabrication.

FIG. 4A is a photograph of the biological tube described in Example 4 prepared using a 50:50 mixture of NHLFs and BSMCs in hyaluronic acid (HA) hydrogel immediately after fabrication.

FIG. 4B is a photograph of the biological tube described in Example 4 on day 1 after fabrication.

FIG. 4C is a photograph of a histological section of the biological tube described in Example 4 stained using the H&E stain on day 1 after fabrication.

FIG. 5A is a photograph of the results of a handling test performed on the biological tubes prepared using HA at a cell density of 300 million cells/mL described in Example 5.

FIG. 5B is a photograph of the results of a suture test performed on the biological tubes prepared using HA at a cell density of 300 million cells/mL described in Example 5.

FIG. 5C is a photograph of an air perfusion test performed on the biological tubes prepared using HA at a cell density of 300 million cells/mL described in Example 5.

FIGS. 6A-6D are photographs of histological sections of the biological tubes described in Example 5 fabricated from 200 million cells/mL with HA (FIG. 6A), 300 cells/mL with HA (FIG. 6B), 200 million cells/mL with collagen (FIG. 6C), and 300 million cells/mL with collagen (FIG. 6D).

FIG. 7A is a graph showing the suture retention data generated using the biological tubes described in Example 5 with the suture retention data for each of the four biological tubes evaluated.

FIG. 7B is a chart showing the suture retention data generated using the biological tubes described in Example 5 with the suture retention data for groups of the biological tubes evaluated, including statistical analysis.

FIGS. 8A and 8B are photographs of histological sections of the bi-layered constructs described in Example 6 fabricated using human pulmonary artery endothelial cells (HPAECs) in the interior layer (the endothelial cells were stained for CD31 in the lumen of the tube).

FIGS. 9A and 9B are photographs of histological sections of the bi-layered constructs described in Example 6 fabricated using human umbilical vein endothelial cells (HUVECs) in the interior layer (the endothelial cells were stained for CD31 in the lumen of the tube).

FIGS. 10A and 10B are photographs of the single-layered biological tube described in Example 10 prepared using a 50:50 mixture of intestinal myofibroblasts (IMFs) and human dermal fibroblasts (HDFs) using HA and gelatin taken immediately after fabrication (FIG. 10A) and one day after fabrication (FIG. 10B).

FIG. 10C is a photograph of a histological section of the single-layered biological tube described in Example 10 stained using the H&E stain.

FIGS. 11A and 11B are photographs taken immediately after fabrication of the double-layered biological tubes described in Example 11 with the second layer a 50:50 mixture of IMFs and MSCs using HA and gelatin. In FIG. 11A, the first layer is alginate and gelatin and in FIG. 11B, the first layer is NOVOGEL® 1.

FIG. 12A is a photograph taken immediately after fabrication of the triple-layered biological tube described in Example 12 with a first layer of NOVOGEL® 1.0, a second layer of a 50:50 mixture of IMFs and HDFs in HA and gelatin, and a third layer of alginate and gelatin cross-linked in calcium chloride.

FIGS. 12B and 12C are photographs of a histological section of the triple-layered biological tube described in Example 12 stained using the H&E stain (FIG. 12B) and Masson's trichrome stain (FIG. 12C).

FIGS. 13A-13C are photographs of the four-layered biological tubes described in Example 13 with a first layer of NOVOGEL® 1.0, a second layer of Caco-2 cells, a third layer of a 50:50 mixture of IMFs and HDFs in HA and gelatin, and a fourth layer of alginate and gelatin cross-linked in calcium chloride taken immediately after fabrication (FIG. 13A), 1 day after fabrication (FIG. 13B), and 10 days after fabrication (FIG. 13C).

FIGS. 14A-14D are photographs of a histological section of the four-layered biological tube described in Example 13. The four-layered biological tubes were stained using the H&E stain three days after fabrication (FIG. 14A) and 10 days after fabrication (FIG. 14B). And, were stained using Masson's trichrome stain 3 days after fabrication (FIG. 14C) and 10 days after fabrication (FIG. 14D).

FIGS. 15A-15C are photographs of the four-layered biological tubes described in Example 14 with a first layer of NOVOGEL® 1.0, a second layer of Caco-2 cells in 0.5% collagen and 5% gelatin, a third layer of a 50:50 mixture of IMFs and MSCs in HA and gelatin, and a fourth layer of alginate and gelatin cross-linked in calcium chloride immediately after fabrication (FIG. 15A), 3 days after fabrication (FIG. 15B), and 10 days after fabrication (FIG. 15C).

FIGS. 16A-16D are photographs of a histological section of the four-layered biological tube described in Example 14. The four-layered biological tubes were stained using the H&E stain three days after fabrication (FIG. 16A) and 10 days after fabrication (FIG. 16B). And, were stained using Masson's trichrome stain 3 days after fabrication (FIG. 16C) and 10 days after fabrication (FIG. 16D).

FIGS. 17A and 17B are photographs of the three-layered biological tubes described in Example 15 with a first layer of NOVOGEL® 1.0, a second layer of IMFs, MSCs, and Caco-2 cells in 0.5% HA and 5% gelatin, and a third layer of alginate and gelatin cross-linked in calcium chloride taken immediately after fabrication (FIG. 17A) and 1 day after fabrication (FIG. 17B).

FIGS. 18A-18D are photographs of a histological section of the three-layered biological tube described in Example 15. The three-layered biological tubes were stained using the H&E stain three days after fabrication (FIG. 18A) and 10 days after fabrication (FIG. 18B). And, were stained using Masson's trichrome stain 3 days after fabrication (FIG. 18C) and 10 days after fabrication (FIG. 18D). As shown in FIGS. 18C and 18D, after ten days, Caco-2 cells have organized along the exterior of the tube.

### DETAILED DESCRIPTION OF THE INVENTION

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

All numbers in this description indicating amounts, ratios or materials, physical properties of materials, and/or use are to be understood as modified by the word “about,” except as otherwise explicitly indicated. As used herein, “about” includes the recited number±10%. Thus, “about ten” means 9 to 11.

As used herein, “consists essentially” means that the specified cell type is the only cell type present, but the bio-ink may contain other non-cellular material including but not limited to extrusion compounds, hydrogels, extracellular matrix components, nutritive and media components, inorganic and organic salts, acids and bases, buffer compounds, and other non-cellular components that promote cell survival, adhesion, growth, or that facilitate bioprinting.

As used herein “exposed” to a “cross-linker” or “cross-linking” agent means non-physiological levels of the specific cross-linker or cross-linking agent, or levels of the specific cross-linking agent that are not sufficient to result in significant crosslinking of a given cross-linkable substance. Significant crosslinking is greater than 1%, 2%, 3%, 4% or 5% crosslinking.

As used herein, “tissue” means an aggregate of cells.

As used herein, “layer” means an association of cells in a sheet. In one embodiment, the sheet is planar, i.e., in X and Y planes. In another embodiment, the layer is tubular. In another embodiment, the layer is at least one cell thick. In some embodiments, the biological tubes described herein include one layer. In other embodiments, the biological tubes described herein include a plurality of layers. In some embodiments, a layer forms a contiguous, substantially contiguous, or non-contiguous sheet of cells. In some embodiments, each layer of a biological tube described herein comprises multiple cells in the X, Y, and Z axes.

As used herein, “bio-ink” means a liquid, semi-solid, or solid composition for use in bioprinting. In some embodiments, bio-ink comprises cell solutions, cell aggregates, cell-comprising gels, multicellular bodies, cellular pastes, or tissues. In some embodiments, the bio-ink additionally comprises non-cellular materials that provide specific biomechanical properties that enable bioprinting. In some embodiments, the bio-ink comprises an extrusion compound. In some embodiments, the extrusion compound is engineered to be removed after the bioprinting process. In some embodiments, at least some portion of the extrusion compound remains entrained with the cells post-printing and is not removed.

As used herein, “bio-compatible liquid” means any liquid capable of contacting or completely covering cells without damage to the cells, examples include but are not limited to growth media and physiological buffers disclosed in this application.

As used herein, “biological construct” means a tube, duct, or passage which carries gas, fluids, or solids in a biological system such as in humans and animals and which comprises living cells. In some embodiments, the biological tube is engineered using the methods disclosed herein. In some embodiments, the “biological construct” is a “biological tube.” The term “biological construct” is used interchangeably with “tissue construct.”

As used herein, “bioprinting” means utilizing three-dimensional, precise deposition of cells (e.g., cell solutions, cell-containing gels, cell suspensions, cell concentrations, multicellular aggregates, multicellular bodies, etc.) via methodology that is compatible with an automated or semi-automated, computer-aided, three-dimensional prototyping device (e.g., a bioprinter). Suitable bioprinters include the NOVOGEN BIOPRINTER® from Organovo, Inc. (San Diego, Calif.) and those described in U.S. Pat. No. 9,149,952 and U.S. Appl. Publ. Nos. 2015/0093932, 2015/0004273, and 2015/0037445. Some bioprinting methods are extrusion methods which comprise forcing a high viscosity bio-ink through an opening for deposition to a surface. Extrusion methods can be continuous or discontinuous. Other bioprinting methods are ejection methods which comprise spraying an aerosol, droplets, or a mist onto a surface. This type of method requires a low-viscosity bio-ink. An example of this method is the technique of ink-jetting. These methods are incompatible with high viscosity bio-inks.

As used herein, the term “engineered,” when used to refer to tissues and/or organs means that cells, cell solutions, cell suspensions, cell-comprising gels or pastes, cell concentrates, multicellular aggregates, and layers thereof are positioned to form three-dimensional structures by a computer-aided device (e.g., a bioprinter) according to a computer script. In some embodiments, the computer script is, for example, one or more computer programs, computer applications, or computer modules. In some embodiments, three-dimensional tissue constructs form through the post-printing fusion of cells or multicellular bodies which, in some cases, is similar to self-assembly phenomena in early morphogenesis.

As used herein, “filament” means a slender threadlike object comprising a bio-ink. Filaments can be wound over a rotating mandrel to create a tube comprising living cells. In some embodiments, the filament has a length of at least 30 mm. In some embodiments, the filament has a length of between about 30 mm and about 5,000 mm.

As used herein, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and not able to be removed from the tissue without damage/destruction of said tissue. In some embodiments, decellularized tissue scaffolds include decellularized native tissues or decellularized cellular material generated by cultured cells in any manner; for example, cell layers that are allowed to die or are decellularized, leaving behind the ECM they produced while living. The term “scaffoldless,” therefore, is intended to imply that pre-formed scaffold is not an integral part of the engineered tissue at the time of use, either having been removed or remaining as an inert component of the engineered tissue. “Scaffoldless” is used interchangeably with “scaffold-free” and “free of pre-formed scaffold.”

As used herein, “subject” means any individual, which is a human, a non-human animal, any mammal, or any vertebrate. The term is interchangeable with “patient,” “recipient,” and “donor.”

As used herein, “test substance” refers to any biological, chemical, or physical substance under evaluation for its ability to elicit a change in a tissue compared to a tissue not treated with said substance. A non-limiting example of a change in a tissue could be an allergic reaction, a toxic reaction, an irritation reaction: a change that is measured by a defined molecular state such as a change in mRNA levels or activity, changes in protein levels, changes in protein modification or epigenetic changes; or a change that results in a measurable cellular outcome such as a change in proliferation, apoptosis, cell viability, cell division, cell motility, cytoskeletal rearrangements, chromosomal number, or composition. Test substances include, but are not limited to: chemical compositions containing an active or inactive ingredient, either in whole, in part, isolated, or purified; physical stressors such as light, UV light, mechanical stress, heat, or cold; biological agents such as bacteria, viruses, parasites, or fungi. “Test substance” also refers to a plurality of substances mixed or applied separately.

In some embodiments, the test substance is an antiviral, an analgesic agent, an antidepressant agent, a diuretic agent, or a proton pump inhibitor. In some embodiments, the test substance is a cytokine, a chemokine, a small molecule drug, a large molecule drug, a protein or a peptide. In some embodiments, the test substance is a chemotherapeutic agent. In some embodiments, the test substance is ibuprofen, acetaminophen, lithium, acyclovir, amphotericin B, and aminoglycoside, a beta lactams, foscavir, ganciclovir, pentamidine, a quinolone, a sulfonamide, vancomycin, rifampin, adefovir, indinavir, didofovir, tenofovir, methotrexate, lansoprazole, omeprazole, pantopraxole, allopurinol, phenytoin, ifosfamide, gentamycin, or zoledronate. In some embodiments, the test substance is radiation. In some embodiments, the test substance is an immune activator or modulator.

As used herein, “use” encompasses a variety of possible uses of the tissue which will be appreciated by one skilled in the art. These uses include by way of non-limiting example: implantation or engraftment of the engineered tissue into or onto a subject; inclusion of the tissue in a biological assay for the purposes of biological, biotechnological, or pharmacological discovery; toxicology testing, including teratogen testing; pharmacology testing, including testing to determine pharmacokinetics and drug metabolism and absorption and penetration, cosmetic testing, including testing to determine sensitization, potential to cause irritation or corrosion of any layer of the dermis, to any test chemical or non-chemical agent including ultraviolet light. “Use” can also refer to the process of maturation, or tissue cohesion, in vitro after bioprinting.

As used herein, a “mandrel” is a cylindrical or conical member that may be hollow or solid. In one embodiment, a mandrel is a cylindrical solid. In another embodiment, a mandrel is a conical solid, i.e., one that narrows in diameter along its length. A mandrel may be made of any bio-compatible material including metal, ceramic, polymer, or glass.

### Bioprinting

Disclosed herein are devices, systems, and methods for fabricating biological tubes. In some embodiments, the devices for fabricating biological tubes are bioprinters. In some embodiments, the methods comprise the use of bioprinting techniques. In some embodiments, the biological tubes fabricated by use of the devices, systems, and methods described herein are bioprinted.

In some embodiments, bioprinted biological tubes are made with a method that utilizes a rapid prototyping technology based on three-dimensional, automated, computer-aided deposition of cells, including cell solutions, cell suspensions, cell-comprising gels or pastes, cell concentrations, multicellular bodies (e.g., cylinders, spheroids, ribbons, etc.), and support material onto a biocompatible surface (e.g., composed of hydrogel and/or a porous membrane) by a three-dimensional delivery device (e.g., a bioprinter).

While a number of methods are available to arrange cells, multicellular aggregates, and/or layers thereof on a biocompatible surface to produce a three-dimensional structure including manual placement, positioning by an automated, computer-aided machine such as a bioprinter is advantageous. Advantages of delivery of cells or multicellular bodies with this technology include rapid, accurate, and reproducible placement of cells or multicellular bodies to produce constructs exhibiting planned or pre-determined orientations or patterns of cells, multicellular aggregates, and/or layers thereof with various compositions. Advantages also include assured high cell density, while minimizing cell damage.

In some embodiments, methods of bioprinting are continuous and/or substantially continuous. A non-limiting example of a continuous bioprinting method is to dispense bio-ink from a bioprinter via a dispense tip (e.g., a syringe, capillary tube, etc.) connected to a reservoir of bio-ink. In some embodiments, a continuous bioprinted method dispenses bio-ink in a layer or a plurality of layers bioprinted adjacently (e.g., stacked) to form a biological tube. In various embodiments, 2, 3, 4, 5, 6, 7, 8, 9, 10, 11, 12, 13, 14, 15, or more layers are bioprinted adjacently (e.g., stacked) to form a biological tube.

Continuous deposition printing provides an advantage in that it enables cells to be placed within a precise geometry and enables the use of multiple bio-ink formulations including, but not limited to, inert gels such as NOVOGEL® 2.0 and NOVOGEL® 3.0, and cell paste. Continuous deposition allows optional incorporation of various biomaterials into the NOVOGEL® formulation and various printing surfaces to promote extracellular matrix production and differentiation.

Methods in continuous bioprinting may involve optimizing and/or balancing parameters such as print height, pump speed, robot speed, or combinations thereof independently or relative to each other. Without limitation a suitable and/or optimal dispense distance does not result in material flattening or adhering to the dispensing needle. The pump speed may be suitable and/or optimal when the residual pressure build-up in the system is low. Favorable pump speeds may depend on the ratio between the cross-sectional areas of the reservoir and dispense needle with larger ratios requiring lower pump speeds. In some embodiments, a suitable and/or optimal print speed enables the deposition of a uniform line without affecting the mechanical integrity of the material.

In some embodiments, at least one layer of cells is deposited by spraying with a bioprinter. In some embodiments, the deposition is by aerosol spraying. Aerosol spray bioprinting techniques allow for the spray of materials that include, for example, a cell suspension, media, bio-ink, or a combination thereof. In some embodiments, the engineered tissues and methodologies described herein highlight the ability to aerosol spray (e.g., spray) single cells at a resolution of one cell layer thickness and the ability to spray cell aggregates. The sprayed layer could, however, also be modified by changing parameters including but not limited to spray material velocity, distance, time, volume, and viscosity.

The aerosol spray method does not require a flat printing surface, such as a transwell membrane, to zero the initial printing position in the x, y, and z-axes. The aerosol spray method is optionally used to apply a layer to an uneven surface such as a structure previously printed by continuous deposition.

While a number of methods are available to arrange cells, multicellular aggregates, and/or layers thereof on a biocompatible surface to produce a three-dimensional structure including manual placement, positioning by an automated, computer-aided machine such as a bioprinter is advantageous. Advantages of delivery of cells or multicellular bodies with this technology include rapid, accurate, and reproducible placement of cells or multicellular bodies to produce constructs exhibiting planned or pre-determined orientations or patterns of cells, multicellular aggregates, and/or layers thereof with various compositions. Advantages also include assured high cell density, while minimizing cell damage.

In some embodiments, deposition of a second bio-ink occurs after deposition of a first bio-ink. In some embodiments, deposition of the second bio-ink is temporally delayed before it is deposited on the first bio-ink. In some embodiments, deposition of a third and subsequent bio-ink layer is temporarily delayed before it is deposited on the previous bio-ink layer. In some embodiments, the delay is between about 20 milliseconds and about 4 weeks, about 20 milliseconds and about 1 week, about 20 milliseconds and about 4 days, about 20 milliseconds and about 1 day, about 20 milliseconds and about 12 hours, about 20 milliseconds and about 6 hours, about 20 milliseconds and about 1 hour, about 20 milliseconds and about 30 minutes, about 20 milliseconds and about 10 minutes, about 20 milliseconds and about 1 minute, about 20 milliseconds and about 30 seconds, about 20 milliseconds and about 1 second, about 20 milliseconds and about 500 milliseconds, about 20 milliseconds and about 100 milliseconds, about 100 milliseconds and about 4 weeks, about 100 milliseconds and about 1 week, about 100 milliseconds and about 4 days, about 100 milliseconds and about 1 day, about 100 milliseconds and about 12 hours, about 100 milliseconds and about 6 hours, about 100 milliseconds and about 1 hour, about 100 milliseconds and about 30 minutes, about 100 milliseconds and about 10 minutes, about 100 milliseconds and about 1 minute, about 100 milliseconds and about 30 seconds, about 100 milliseconds and about 1 second, about 100 milliseconds and about 500 milliseconds, about 500 milliseconds and about 4 weeks, about 500 milliseconds and about 1 week, about 500 milliseconds and about 4 days, about 500 milliseconds and about 1 day, about 500 milliseconds and about 12 hours, about 500 milliseconds and about 6 hours, about 500 milliseconds and about 1 hour, about 500 milliseconds and about 30 minutes, about 500 milliseconds and about 10 minutes, about 500 milliseconds and about 1 minute, about 500 milliseconds and about 30 seconds, about 500 milliseconds and about 1 second, about 1 second and about 4 weeks, about 1 second and about 1 week, about 1 second and about 4 days, about 1 second and about 1 day, about 1 second and about 12 hours, about 1 second and about 6 hours, about 1 second and about 1 hour, about 1 second and about 30 minutes, about 1 second and about 10 minutes, about 1 second and about 1 minute, about 1 second and about 30 seconds, about 30 seconds and about 4 weeks, about 30 seconds and about 1 week, about 30 seconds and about 4 days, about 30 seconds and about 1 day, about 30 seconds and about 12 hours, about 30 seconds and about 6 hours, about 30 seconds and about 1 hour, about 30 seconds and about 30 minutes, about 30 seconds and about 10 minutes, about 30 seconds and about 1 minute, about 1 minute and about 4 weeks, about 1 minute and about 1 week, about 1 minute and about 4 days, about 1 minute and about 1 day, about 1 minute and about 12 hours, about 1 minute and about 6 hours, about 1 minute and about 1 hour, about 1 minute and about 30 minutes, about 1 minute and about 10 minutes, about 10 minutes and about 4 weeks, about 10 minutes and about 1 week, about 10 minutes and about 4 days, about 10 minutes and about 1 day, about 10 minutes and about 12 hours, about 10 minutes and about 6 hours, about 30 seconds and about 1 hour, about 10 minutes and about 30 minutes, about 1 hour and about 4 weeks, about 1 hour and about 1 week, about 1 hour and about 4 days, about 1 hour and about 1 day, about 1 hour and about 12 hours, about 1 hour and about 6 hours, about 6 hours and about 4 weeks, about 6 hours and about 1 week, about 6 hours and about 4 days, about 6 hours and about 1 day, about 6 hours and about 12 hours, about 12 hours and about 4 weeks, about 12 hours and about 1 week, about 12 hours and about 4 days, about 12 hours and about 1 day, about 1 day and about 4 weeks, about 1 day and about 1 week, about 1 day and about 4 days, about 4 days and about 4 weeks, about 4 days and about 1 week, or about 1 week and 4 weeks.

### Bioprinter

Disclosed herein, in some embodiments, are bioprinters for fabricating biological tubes. In some embodiments, a bioprinter is any instrument that automates a bioprinting process. In certain embodiments, a bioprinter disclosed herein comprises: a printer head, wherein the printer head comprises a reservoir containing a bio-ink and a deposition orifice, wherein said bio-ink comprises a filament comprising living cells; a calibration element for determining the position of the deposition orifice; an extrusion element for extruding the bio-ink through the deposition orifice; a rotating mandrel for receiving the extruded bio-ink, a motor for rotating the mandrel; and a programmable computer processor.

In some embodiments, a bioprinter dispenses bio-ink in pre-determined geometries (e.g., positions, patterns, etc.) in two or three dimensions. In some embodiments, a bioprinter achieves a particular geometry by moving a printer head relative to a rotating mandrel adapted to receive bioprinted materials. In some embodiments, a bioprinter achieves a particular geometry by moving a rotating mandrel relative to a printer head. In some embodiments, the bioprinter is maintained in a sterile environment.

In some embodiments, a bioprinter disclosed herein comprises one or more printer heads. In some embodiments, a printer head comprises a means for receiving and holding at least one cartridge. As used herein, a “cartridge” refers to any object that is capable of receiving and holding a bio-ink. In some embodiments, a printer head comprises a means for receiving and holding more than one cartridge. In some embodiments, the means for receiving and holding at least one cartridge is selected from: magnetic attraction, a collet chuck grip, a ferrule, a nut, a barrel adapter, or a combination thereof. In some embodiments, the means for receiving and holding at least one cartridge is a collet chuck grip.

In some embodiments, the printer head comprises a deposition orifice. Many shapes are suitable for the deposition orifice. In some embodiments, the shape of the deposition orifice is circular, ovoid, triangular, square, rectangular, polygonal, or irregular. In some embodiments, the shape of the deposition orifice is circular. In some embodiments, the shape of the deposition orifice is square. In some embodiments, the deposition orifice dispenses bio-ink as a filament. In some embodiments, the deposition orifice dispenses bio-ink as a continuous filament. In some embodiments, the dispensing of the filament is by extrusion.

In some embodiments, bio-ink is dispensed by extrusion from a deposition orifice via a dispense tip (e.g., a syringe, needle, capillary tube, etc.) connected to a reservoir of bio-ink. In some embodiments, bio-ink is dispensed by extrusion from a deposition orifice via a dispense tip (e.g., a syringe, needle, capillary tube, etc.) connected to a cartridge of bio-ink.

In some embodiments, the inner diameter of the deposition orifice is between about 10 μm and about 5,000 μm, about 10 μm and about 2,000 μm, about 10 μm and about 1,000 μm, about 10 μm and about 500 μm, about 10 μm and about 100 μm, about 10 μm and about 50 μm, about 50 μm and about 5,000 μm, about 50 μm and about 2,000 μm, about 50 μm and about 1,000 μm, about 50 μm and about 500 μm, about 50 μm and about 100 μm, about 100 μm and about 5,000 μm, about 100 μm and about 2,000 μm, about 100 μm and about 1,000 μm, about 100 μm and about 500 μm, about 500 μm and about 5,000 μm, about 500 μm and about 2,000 μm, about 500 μm and about 1,000 μm, 1,000 μm and about 5,000 μm, about 1,000 μm and about 2,000 μm, or about 2,000 μm and about 5,000 μm.

In some embodiments, a bioprinter disclosed herein comprises a means for calibrating the position of the deposition orifice. In some embodiments, the means for calibrating the position of the deposition orifice is a calibration element. In some embodiments, the calibration element is selected from: laser alignment, optical alignment, mechanical alignment, piezoelectric alignment, magnetic alignment, electrical field or capacitance alignment, ultrasound alignment, or a combination thereof. In some embodiments, the calibration element is laser alignment.

In some embodiments, the bioprinter comprises a means for dispensing the bio-ink through the deposition orifice. In some embodiments, the means for dispensing the bio-ink through the deposition orifice is an extrusion element. In some embodiments, the extrusion element extrudes the bio-ink by application of a piston, application of pressure, application of compressed gas, application of hydraulics, or application of a combination thereof. In some embodiments, the extrusion element extrudes the bio-ink through the deposition orifice by application of a piston. In some embodiments, the diameter of the piston is less than the diameter of the deposition orifice.

In some embodiments, the extrusion element is controlled by a programmable computer processor communicatively coupled to the extrusion element.

In some embodiments, the extrusion element dispenses by extrusion the bio-ink at a rate of between about 0.02 mm/s and about 20 mm/s, about 0.02 mm/s and about 10 mm/s, about 0.02 mm/s and about 5 mm/s, about 0.02 mm/s and about 1 mm/s, about 0.02 mm/s and about 0.1 mm/s, about 0.02 mm/s and about 0.05 mm/s, about 0.05 mm/s and about 20 mm/s, about 0.05 mm/s and about 10 mm/s, about 0.05 mm/s and about 5 mm/s, about 0.05 mm/s and about 1 mm/s, about 0.05 mm/s and about 0.1 mm/s, about 0.1 mm/s and about 20 mm/s, about 0.1 mm/s and about 10 mm/s, about 0.1 mm/s and about 5 mm/s, about 0.1 mm/s and about 1 mm/s, about 1 mm/s and about 20 mm/s, about 1 mm/s and about 10 mm/s, about 1 mm/s and about 5 mm/s, about 5 mm/s and about 20 mm/s, about 5 mm/s and about 10 mm/s, or about 10 mm/s and 20 mm/s. In some embodiments, the extrusion element dispenses by extrusion the bio-ink at a rate of between about 0.02 mm/s and about 0.05 mm/s.

In some embodiments, the extrusion element dispenses by extrusion the bio-ink at a rate of between about 0.1 μL/s and about 34 μL/s, about 0.1 μL/s and about 20 μL/s, about 0.1 μL/s and about 10 μL/s, about 0.1 μL/s and about 5 μL/s, about 0.1 μL/s and about 1 μL/s, about 0.1 μL/s and about 0.5 μL/s, about 0.5 μL/s and about 34 μL/s, about 0.5 μL/s and about 20 μL/s, about 0.5 μL/s and about 10 μL/s, about 0.5 μL/s and about 5 μL/s, about 0.5 μL/s and about 1 μL/s, about 1 μL/s and about 34 μL/s, about 1 μL/s and about 20 μL/s, about 1 μL/s and about 10 μL/s, about 1 μL/s and about 5 μL/s, about 5 μL/s and about 34 μL/s, about 5 μL/s and about 20 μL/s, about 5 μL/s and about 10 μL/s, about 10 μL/s and about 34 μL/s, about 10 μL/s and about 20 μL/s, or about 20 μL/s and about 34 μL/s. In some embodiments, the extrusion element dispenses the bio-ink by extrusion at a rate of between about 0.1 μL and about 0.5 μL.

In some embodiments, the bioprinter comprises a positioning element for positioning the printer head relative to the mandrel and translating the position of the printer head across the length of the mandrel.

In some embodiments, the positioning element is controlled by a programmable computer processor communicatively coupled to the extrusion element.

In some embodiments, the positioning element translates the position of the printer head at a rate of between about 0.05 mm/s and about 1 mm/s, about 0.05 mm/s and about 0.8 mm/s, about 0.05 mm/s and about 0.5 mm/s, about 0.05 mm/s and about 0.3 mm/s, about 0.05 mm/s and about 0.2 mm/s, about 0.05 mm/s and about 0.1 mm/s, about 0.1 mm/s and about 1 mm/s, about 0.1 mm/s and about 0.8 mm/s, about 0.1 mm/s and about 0.5 mm/s, about 0.1 mm/s and about 0.3 mm/s, about 0.1 mm/s and about 0.2 mm/s, about 0.2 mm/s and about 1 mm/s, about 0.2 mm/s and about 0.8 mm/s, about 0.2 mm/s and about 0.5 mm/s, about 0.2 mm/s and about 0.3 mm/s, about 0.3 mm/s and about 1 mm/s, about 0.3 mm/s and about 0.8 mm/s, about 0.3 mm/s and about 0.5 mm/s, about 0.5 mm/s and about 1 mm/s, about 0.5 mm/s and about 0.8 mm/s, or about 0.8 mm/s and about 1 mm/s. In some embodiments, the positioning element translates the position of the printer head at a rate of between about 0.03 mm/s and about 0.25 mm/s.

In some embodiments, the bioprinter comprises a receiving surface. In some embodiments, the receiving surface is a mandrel. In some embodiments, the receiving surface is a rotating mandrel. In some embodiments, the surface of the mandrel is flat or substantially flat. In some embodiments, the surface of the mandrel is smooth or substantially smooth. In some embodiments, the surface of the mandrel is both substantially flat and substantially smooth.

In some embodiments, the bioprinter comprises a motor for rotating the mandrel. In some embodiments, the motor is controlled by a programmable computer processor communicatively coupled to the motor.

In some embodiments, the motor rotates the mandrel at a rate of between about 1 rpm and about 200 rpm, about 1 rpm and about 150 rpm, about 1 rpm and about 100 rpm, about 1 rpm and about 50 rpm, about 1 rpm and about 30 rpm, about 1 rpm and about 20 rpm, about 1 rpm and about 10 rpm, about 1 rpm and about 5 rpm, about 5 rpm and about 200 rpm, about 5 rpm and about 150 rpm, about 5 rpm and about 100 rpm, about 5 rpm and about 50 rpm, about 5 rpm and about 30 rpm, about 5 rpm and about 20 rpm, about 5 rpm and about 10 rpm, 10 rpm and about 200 rpm, about 10 rpm and about 150 rpm, about 10 rpm and about 100 rpm, about 10 rpm and about 50 rpm, about 10 rpm and about 30 rpm, about 10 rpm and about 20 rpm, about 20 rpm and about 200 rpm, about 20 rpm and about 150 rpm, about 20 rpm and about 100 rpm, about 20 rpm and about 50 rpm, about 20 rpm and about 30 rpm, about 30 rpm and about 200 rpm, about 30 rpm and about 150 rpm, about 30 rpm and about 100 rpm, about 30 rpm and about 50 rpm, about 50 rpm and about 200 rpm, about 50 rpm and about 150 rpm, about 50 rpm and about 100 rpm, about 100 rpm and about 200 rpm, about 100 rpm and about 150 rpm, or about 150 rpm and about 200 rpm. In some embodiments, the motor rotates the mandrel at a rate of between about 10 rpm and about 29 rpm. In some embodiments, the motor rotates the mandrel at a rate of about 100 rpm. In some embodiments, the motor rotates the mandrel at a rate of about 17 rpm.

In some embodiments, a bioprinter disclosed herein further comprises a means for adjusting temperature. In some embodiments, the means for adjusting temperature adjusts and/or maintains the ambient temperature. In some embodiments, the means for adjusting temperature adjusts and/or maintains the temperature of the printer head, reservoir, contents of the reservoir (e.g., a bio-ink), or the mandrel.

In some embodiments, the means for adjusting temperature is a heating element. In some embodiments, the means for adjusting temperature is a heater. In some embodiments, the means for adjusting temperature is a radiant heater, a convection heater, a conductive heater, a fan heater, a heat exchanger, or a combination thereof. In some embodiments, the means for adjusting temperature is a cooling element. In some embodiments, the means for adjusting temperature is a container of coolant, a chilled liquid, ice, or a combination thereof. In some embodiments, the means for adjusting temperature is a radiant cooler, convection cooler, a conductive cooler, a fan cooler, or a combination thereof.

In some embodiments, the means for adjusting temperature adjusts a temperature to about 0, 5, 10, 15, 20, 25, 30, 35, 40, 45, 50, 55, 60, 65, 70, 75, 80, 85, or 90° C. In some embodiments, temperature is adjusted to between about 40° C. and about 90° C. In some embodiments, temperature is adjusted to between about 0° C. and about 10° C.

In some embodiments, the bioprinter further comprises a means for applying a wetting agent to one or more of the mandrel, the deposition orifice, the reservoir, the bio-ink, or the printed construct. In some embodiments, the means for applying the wetting agent is any suitable method of applying a fluid (e.g., a sprayer, a pipette, an inkjet, etc.). In some embodiments, the wetting agent is water, tissue culture media, buffered salt solutions, serum, or a combination thereof. In some embodiments, a wetting agent is applied after the bio-ink is dispensed by the bioprinter. In some embodiments, a wetting agent is applied simultaneously or substantially simultaneously with the bio-ink being dispensed by the bioprinter. In some embodiments, a wetting agent is applied prior to the bio-ink being dispensed by the bioprinter.

### Printer Head

Disclosed herein are bioprinters for fabricating biological tubes. In some embodiments, a bioprinter comprises one or more printer heads. In some embodiments, a printer head comprises a means for receiving and holding at least one reservoir. In some embodiments, the printer head further comprises a cartridge. In some embodiments, the reservoir supplies bio-ink to at least one cartridge. In some embodiments, a printer head attaches at least one cartridge to a bioprinter.

Many means for receiving and holding at least one cartridge are suitable. Suitable means for receiving and holding at least one cartridge include those that reliably, precisely, and securely attach at least one cartridge to a bioprinter. In some embodiments, the means for receiving and holding at least one cartridge is, by way of non-limiting example, magnetic attraction, a collet chuck grip, a ferrule, a nut, a barrel adapter, or a combination thereof.

In some embodiments, a printer head disclosed herein receives and holds one cartridge. In various other embodiments, a printer head disclosed herein receives and holds 2, 3, 4, 5, 6, 7, 8, 9, 10, or more cartridges simultaneously. In some embodiments, a printer head disclosed herein further comprises a means to select a cartridges to be employed in bioprinting from among a plurality of cartridges received and held.

In some embodiments, a printer head disclosed herein comprises (or is in fluid communication with) a reservoir containing bio-ink beyond the capacity of one or more cartridges. In some embodiments, a reservoir supplies bio-ink to one or more cartridges for dispensing via a deposition orifice. Printer head configurations including a reservoir are particularly useful in continuous or substantially continuous bioprinting applications. Many volumes are suitable for a reservoir disclosed herein. In various embodiments, a reservoir has an internal volume of between about 1 mL and about 500 mL, about 1 mL and about 200 mL, about 1 mL and about 100 mL, about 1 mL and about 50 mL, about 1 mL and about 10 mL, about 1 mL and about 5 mL, about 5 mL and about 500 mL, about 5 mL and about 200 mL, about 5 mL and about 100 mL, about 5 mL and about 50 mL, about 5 mL and about 10 mL, about 10 mL and about 500 mL, about 10 mL and about 200 mL, about 10 mL and about 100 mL, about 10 mL and about 50 mL, about 50 mL and about 500 mL, about 50 mL and about 200 mL, about 50 mL and about 100 mL, about 100 mL and about 500 mL, about 100 mL and about 200 mL, and about 200 mL and about 500 mL.

In some embodiments, bioprinting involves using a computer to configure parameters such as print height, pump speed, robot speed, or combinations thereof independently or relative to each other. In some embodiments, computer code specifies the positioning of a printer head to configure printer head height above a receiving surface. In some embodiments, computer code specifies the direction and speed of the motion of a printer head to configure dispensing characteristics for bio-ink.

In some embodiments, the bioprinter comprises a programmable computer processor communicatively connected to the calibration element, the extrusion element, and the motor. In some embodiments, the programmable computer processor regulates motion of the printer head, regulates extrusion of the bio-ink, and/or regulates the rotation of the mandrel.

### Rotating Mandrel

Provided herein is a bioprinter apparatus for fabricating biological tubular structures including vascular, pulmonary, and other tubular structures. In some embodiments, a bioprinter apparatus facilitates fabricating extra-long (e.g., >30 mm) biological tubular structures. In some embodiments, the bioprinter comprises: a printer head, wherein the printer head comprises a reservoir containing a bio-ink and a deposition orifice, wherein said bio-ink comprises a filament comprising living cells; a calibration element for determining the position of the deposition orifice; an extrusion element for extruding the bio-ink through the deposition orifice; a rotating mandrel for receiving the extruded bio-ink, a motor for rotating the mandrel; and a programmable computer processor. In some embodiments, the bioprinter further comprises a collet and a base plate. In some embodiments, the calibration element, the extrusion element, and/or the motor for rotating mandrel is computer-controlled. In some embodiments, the calibration element, the extrusion element, and/or the motor for rotating mandrel is optionally manually-controlled.

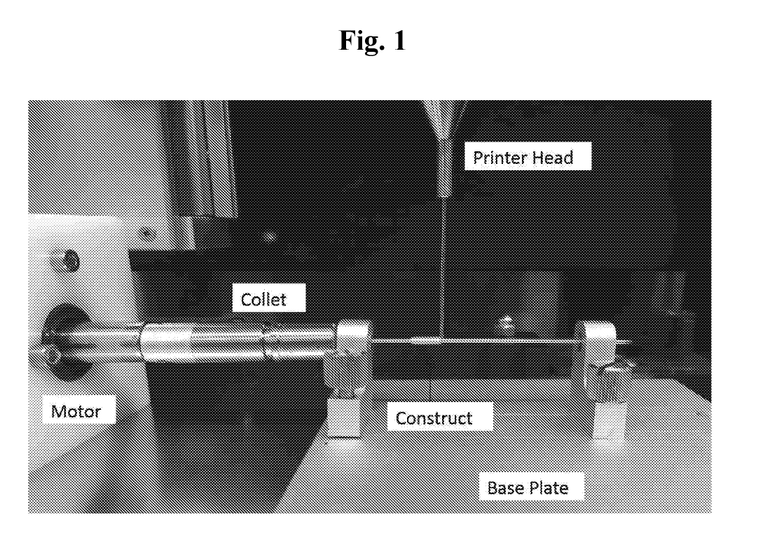

In some embodiments, the bioprinter apparatus comprises a motor connected to a collet that reversibly holds a mandrel situated between two oversized rotatable bearings (see FIG. 1). Many types of small motors are suitable, including by way of non-limiting examples, DC stepper motors, DC servo motors, and AC motors, possessing rotational rates of 0-100 revolutions per minute. The rotating mandrel is optionally constructed of metal such as stainless steel, ceramic, Teflon, plastics such as polyester based polymers, polypropylene, poly-vinyl, rigid hydrogels, hollow sleeves filled with liquid (providing a hydrostatic skeleton), and the like, including combinations thereof. In some embodiments, the mandrel is degradable. In some embodiments, the mandrel is hollow. In some embodiments, the mandrel is solid. In some embodiments, the mandrel is partially hollow and partially solid. In some embodiments, the mandrel is tapered. In some embodiments, the mandrel is non-tapered.

In some embodiments, the mandrel is porous. In some embodiments, the mandrel is permeable to gases, liquids, or combinations thereof. In some embodiments, the mandrel is non-porous.

In some embodiments, the mandrel is removable. Removability of the mandrel facilitates culture and maturation of the fabricated tubular construct by allowing the construct to be cultured while still on the mandrel.

In some embodiments, the mandrel has an outer diameter of between about 0.1 mm and about 200 mm, about 0.1 mm and about 100 mm, about 0.1 mm and about 50 mm, about 0.1 mm and about 10 mm, about 0.1 mm and about 1 mm, about 1 mm and about 200 mm, about 1 mm and about 100 mm, about 1 mm and about 50 mm, about 1 mm and about 10 mm, about 10 mm and about 200 mm, about 10 mm and about 100 mm, about 10 mm and about 50 mm, about 50 mm and about 200 mm, about 50 mm and about 100 mm, or about 100 mm and about 200 mm. In some embodiments, the mandrel has an outer diameter of between about 1 mm and about 120 mm. In some embodiments, the mandrel has an outer diameter of between about 1 mm and about 3.2 mm.

Mandrels with an outer diameter of between about 1 mm and about 3.2 mm support the production of tubular constructs with internal diameters of about the same diameter at the time-point of removal from the mandrel. Tissue contraction typically reduces the internal diameter as the tissue further matures. The extent of contraction will vary with factors such as cellular composition, hydrogel composition, and maturation conditions.

In some embodiments, the mandrel has a length of between about 50 mm and about 500 mm, about 50 mm and about 200 mm, about 50 mm and about 100 mm, about 100 mm and about 500 mm, about 100 mm and about 200 mm, or about 200 mm and about 500 mm.

The linear surface velocity of the mandrel is calculated by Formula (I):

surface velocity=(mandrel diameter (mm)*π*motor rotation (rpm))/60 seconds

Linear surface velocities for mandrels having outer diameters between 1 mm and 120 mm are shown in Table 1.

TABLE 1

| Mandrel Outer | mandrel rotation at | mandrel rotation at |
|---|---|---|
| Diameter (mm) | 10 rpm (rev/min) | 29 rpm (rev/min) |
| 1 | 0.52 | 1.52 |
| 2 | 1.05 | 3.04 |
| 3 | 1.57 | 4.56 |
| 10 | 5.24 | 15.19 |
| 20 | 10.47 | 30.37 |
| 30 | 15.71 | 45.56 |
| 100 | 52.37 | 151.86 |
| 120 | 62.84 | 182.24 |

In some embodiments, the surface velocity of the mandrel is between about 0.05 mm/sec to about 300 mm/sec, about 0.05 mm/sec to about 150 mm/sec, about 0.05 mm/sec to about 76 mm/sec, about 0.05 mm/sec to about 15 mm/sec, about 0.05 mm/sec to about 1.5 mm/sec, about 1.5 mm/sec to about 300 mm/sec, about 1.5 mm/sec to about 150 mm/sec, about 1.5 mm/sec to about 76 mm/sec, about 1.5 mm/sec to about 15 mm/sec, about 15 mm/sec to about 300 mm/sec, about 15 mm/sec to about 150 mm/sec, about 15 mm/sec to about 76 mm/sec, about 76 mm/sec to about 300 mm/sec, about 15 mm/sec to about 150 mm/sec, or about 150 mm/sec to about 303 mm/sec. In some embodiments, the surface velocity of the mandrel is between about 0.5 mm/sec and about 180 mm/sec.

In some embodiments, the mandrel is fitted with a form-fitting sheath, with or without porosity. In some embodiments, the mandrel is completely covered with a removable sheath. In some embodiments, the mandrel is partially covered with a removable sheath. The sheath further facilitates culture and maturation of the fabricated tubular construct by allowing the construct to be removed from the mandrel and cultured without destruction prior to maturation. In some embodiments, the sheath comprises a polymer. In some embodiments, the sheath is non-degradable. In some embodiments, the sheath is non-porous. In some embodiments, the sheath is porous. In some embodiments, the sheath is porous such that it is permeable to gases and/or liquids, such as water.

In some embodiments, the sheath has a wall thickness of between about 50 μm and about 5 mm, about 50 μm and about 1 mm, about 50 μm and about 0.5 mm, about 50 μm and about 0.1 mm, about 0.1 mm and about 5 mm, about 0.1 mm and about 1 mm, about 0.1 mm and about 0.5 mm, about 0.5 mm and about 5 mm, about 0.5 mm and about 1 mm, or about 1 mm and about 5 mm. In some embodiments, the sheath has a wall thickness of between about 50 μm and about 5 mm. In some embodiments, the sheath has a wall thickness of between about 50 μm and about 1 mm.

In some embodiments, the rotating mandrel assembly is linked to a printer head in fluid communication with a reservoir containing bio-ink. See, e.g., FIG. 1. The printer head deposits solid or semi-solid aggregated cell paste (cell/hydrogel mixture) or bio-ink onto the surface of the rotating mandrel using continuous deposition or capillary deposition.

The rotation of the mandrel and translation rate of the printer head in relation to the mandrel are coordinated with extrusion rate of the cell paste or bio-ink. If the extrusion rate is too fast, cellular material builds up non-uniformly on the mandrel. Conversely, if the extrusion rate is too slow, stretching and sometimes fracture of the cellular filament is observed.

In some embodiments, the bio-printer apparatus comprises a positioning element for positioning the printer head relative to the mandrel and translating the position of the printer head across the length of the mandrel. In some embodiments, the positioning element translates the position of the printer head at a rate of about 0.03 mm/s to about 0.25 mm/s. In a high-throughput mode, the printer head deposits a cell/hydrogel mixture (cell paste) or bio-ink filaments along the rotating axis of the mandrel forming a tube 30 mm long in about 3.5 minutes (see Table 2).

TABLE 2

| | Mandrel Method |
|---|---|
| Cells | Cell suspension |
| Print time (per tubule) | 3.5 minutes |
| Long (40 mm) tubes created | 8-9 per syringe reservoir |
| Maturation method | Rotation or Bioreactor |

After the printing process is complete, in some embodiments, the mandrel is removed from the collet and cultured in a Petri dish or on a rotator. In some embodiments, the deposited bio-ink is maintained in culture on the mandrel for about 4 hours to about 12 hours. In some embodiments, once cells in the aggregated cell paste or bio-ink fuse, the tube is removed from the mandrel using forceps and the tube can be cultured (e.g., in a rotator, transluminal flow bioreactor, etc.). In some embodiments, the biological tube is maintained in culture for about 8 hours to about 2 weeks.

### Methods and Systems for Calibrating the Position of a Bioprinter Printer Head

Disclosed herein, in certain embodiments, are bioprinters for fabricating tissue constructs. In some embodiments, a reservoir attached to the bioprinter comprises a bio-ink. In some embodiments, the bioprinter deposits the bio-ink in a specific pattern and at specific positions in order to form a specific tissue construct. In some embodiments, the bioprinter further comprises at least one cartridge. In some embodiments, the reservoir supplies bio-ink to at least one cartridge. In some embodiments, a cartridge comprising bio-ink is disposable. In some embodiments, the cartridge is ejected from the bioprinter following extrusion, dispensing, or deposition of the contents. In some embodiments, a new cartridge is attached to the bioprinter.

In order to fabricate complex structures, the bioprinters disclosed herein dispense bio-ink from a reservoir and/or a cartridge with a suitable repeatable accuracy. In various embodiments, suitable repeatable accuracies include those of about ±5, 10, 20, 30, 40, or 50 μm on any axis. In some embodiments, the bioprinters disclosed herein dispense bio-ink from a reservoir and/or a cartridge with a repeatable accuracy of about ±20 μm. However, in some embodiments, due to the removal and insertion of cartridges, the position of the printer head (and thus the cartridges) with respect to the tissue construct varies. Thus, there is a need for a method of precisely calibrating the position of the printer head, cartridge, and dispensing orifice with respect to the printer stage, mandrel, and tissue construct.

In some embodiments, the method of calibrating the position of a printer head comprises use of at least one laser. In further embodiments, the method of calibrating the position of a printer head comprises use of a first and second laser.

In some embodiments, the method of calibrating the position of a printer head comprises manual (e.g., visual) calibration. In some embodiments, the method of calibrating the position of a printer head comprises manual calibration and laser calibration.

In some embodiments, the position of the printer head is calibrated along one axis, wherein the axis is selected from the x-axis, the y-axis, and the z-axis. In some embodiments, the position of the printer head is calibrated along two axes, wherein the axes are selected from the x-axis, the y-axis, and the z-axis. In some embodiments, the position of the printer head is calibrated along three axes, wherein the axes are selected from the x-axis, the y-axis, and the z-axis.

In some embodiments, calibration is made by use of at least one laser. In further embodiments, calibration is made by use of a first and a second laser.

### Method for Calibrating Using a Horizontal Laser

Disclosed herein are methods of calibrating the position of a printer head comprising a dispensing orifice. In some embodiments, a method disclosed herein further comprises activating a laser and generating at least one substantially stable and/or substantially stationary laser beam, and where said laser beam is horizontal to the ground.

In some embodiments, the methods comprise, calibrating the position of a printer head along at least one axis, wherein the axis is selected from the x-axis, y-axis, and z-axis. In some embodiments, the methods comprise calibrating the position of the printer head along at least two axes, wherein the axis is selected from the x-axis, y-axis, and z-axis. In some embodiments, the methods comprise calibrating the position of the printer head along at least three axes, wherein the axis is selected from the x-axis, y-axis, and z-axis. In some embodiments, the methods comprise (a) calibrating the position of the printer head along the y-axis; (b) calibrating the position of the printer head along the x-axis; and/or (c) calibrating the position of the printer head along the z-axis; wherein each axis corresponds to the axis of the same name in the Cartesian coordinate system. In some embodiments, calibration is made by use of at least one laser. In some embodiments, calibration is made by use of a first and a second laser.

In some embodiments, calibrating the position of a printer head along the y-axis comprises: (a) positioning the printer head so that the printer head is (i) located in a first y octant and (ii) the dispensing orifice is below the upper threshold of the laser beam; (b) moving the printer head towards the laser beam and stopping said movement as soon as the laser beam is interrupted by the printer head, wherein the position at which the laser beam is interrupted by the printer head is the first y position; (c) re-positioning the printer head so that the printer head is located in the second y octant and the dispensing orifice is below the upper threshold of the laser beam; (d) moving the printer head towards the laser beam and stopping said movement as soon as the laser beam is interrupted by the printer head, wherein the position at which the laser beam is interrupted is the second y position; (e) and calculating the mid-point between the first y position and the second y position.

In some embodiments, calibrating the position of a printer head along the x-axis comprises: (a) positioning the printer head (i) at the mid-point between the first y position and the second y position, and (ii) outside the sensor threshold of the laser; and (b) moving the printer head towards the sensor threshold and stopping said movement as soon as the printer head contacts the sensor threshold; wherein the position at which the printer head contacts the sensor increased by half the printer head width is the x position.

In some embodiments, calibrating the position of a printer head along the y-axis comprises: (a) positioning the printer head so that the laser beam can measure the precise location of one side of the printer head; (b) positioning the printer head so that the laser beam can measure the precise location of the opposing side of the printer head; (c) and calculating the midpoint location of the printer head to be relative to the laser location during each measurement and the measured distances.

In some embodiments, calibrating the position of a printer head along the x-axis comprises: (a) positioning the printer head so that the laser beam can measure the precise location of one side of the printer head; (b) positioning the printer head so that the laser beam can measure the precise location of the opposing side of the printer head; (c) and calculating the midpoint location of the printer head to be relative to the laser location during each measurement and the measured distances.

In some embodiments, calibrating the position of a printer head along the z-axis comprises: (a) positioning the printer head so that the dispensing orifice is located above the laser beam; and (b) moving the printer head towards the laser beam and stopping said movement as soon as the laser beam is interrupted by the printer head, wherein the position at which the laser beam is interrupted is the z position.

### Method for Calibrating Using a Vertical Laser

Disclosed herein are methods of calibrating the position of a printer head comprising a dispensing orifice. In some embodiments, a method disclosed herein further comprises activating the laser and generating at least one substantially stable and/or substantially stationary laser beam, and where said laser beam is vertical to the ground.

In some embodiments, the methods comprise, calibrating the position of a printer head along at least one axis, wherein the axis is selected from the x-axis, y-axis, and z-axis. In some embodiments, the methods comprise calibrating the position of a printer head along at least two axes, wherein the axis is selected from the x-axis, y-axis, and z-axis. In some embodiments, the methods comprise calibrating the position of a printer head along at least three axes, wherein the axis is selected from the x-axis, y-axis, and z-axis.

In some embodiments, the methods comprise (a) calibrating the position of the printer head along the y-axis; (b) calibrating the position of the printer head along the x-axis; and (c) calibrating the position of the printer head along the z-axis; wherein each axis corresponds to the axis of the same name in the Cartesian coordinate system.

In some embodiments, calibrating the position of a printer head along the y-axis comprises: (a) positioning the printer head so that the printer head is (i) located in a first y octant and (ii) the dispensing orifice is outside the sensor threshold of the laser; (b) moving the printer head towards the laser beam and stopping said movement as soon as the laser beam is interrupted by the printer head, wherein the position at which the laser beam is interrupted by the printer head is the first y position; (c) re-positioning the printer head so that the printer head is located in the second y octant and the dispensing orifice is outside the sensor threshold of the laser; (d) moving the printer head towards the laser beam and stopping said movement as soon as the laser beam is interrupted by the printer head, wherein the position at which the laser beam is interrupted is the second y position; (e) and calculating the mid-point between the first y position and the second y position.

In some embodiments, calibrating the position of a printer head along the x-axis comprises: (a) positioning the printer head (i) at the mid-point between the first y position and the second y position, and (ii) outside the sensor threshold of the laser; and (b) moving the printer head towards the sensor threshold and stopping said movement as soon as the printer head contacts the sensor threshold; wherein the position at which the printer head contacts the sensor increased by half the printer head width is the x position.

In some embodiments, calibrating the position of a printer head along the z-axis comprises: (a) positioning the printer head so that the dispensing orifice is located above the laser beam so that it is just outside of the laser sensor range threshold; and (b) lowering the printer head until the sensor threshold is reached, wherein the position at which the laser sensor threshold is reached is the z position. In some embodiments, steps (a) and (b) are repeated at multiple points of the printer head and measured heights are averaged to determine the z position.

In some embodiments, calibrating the position of a printer head along the z-axis comprises: (a) positioning the printer head so that the laser beam can measure the precise location of one or more points on the bottom of the printer head; (b) calculating the absolute or average location of the printer head based on the laser position and known measured distance.

### Method for Calibrating Using a Vertical and Horizontal Laser

Disclosed herein are methods of calibrating the position of a printer head comprising a dispensing orifice, wherein the printer head is attached to a bioprinter, comprising calibrating the position of the printer head along at least one axis, wherein the axis is selected from the x-axis, y-axis, and z-axis. In some embodiments, the method comprises calibrating the position of a printer head along at least two axes, wherein the axis is selected from the x-axis, y-axis, and z-axis. In some embodiments, the method comprises calibrating the position of a printer head along at least three axes, wherein the axis is selected from the x-axis, y-axis, and z-axis.

In some embodiments, the methods comprise (a) calibrating the position of the printer head along the y-axis; (b) calibrating the position of the printer head along the x-axis; and (c) calibrating the position of the printer head along the z-axis; wherein each axis corresponds to the axis of the same name in the Cartesian coordinate system.

In some embodiments, calibration comprises use of a first laser and a second laser. In some embodiments, the first laser is a vertical laser and the second laser is a horizontal laser.

### System for Calibrating Using a Laser

Disclosed herein are systems for calibrating the position of a cartridge comprising a deposition orifice, wherein the cartridge is attached to a bioprinter, said system comprising: a means for calibrating the position of the cartridge along at least one axis, wherein the axis is selected from the y-axis, x-axis, and z-axis.

Also disclosed herein, in certain embodiments, are systems for calibrating the position of a printer head comprising a dispensing orifice, wherein the printer head is attached to a bioprinter, said system comprising: a means for calibrating the position of the printer head along an x-axis; a means for calibrating the position of the printer head along a y-axis; and a means for calibrating the position of the printer head along a z-axis.

In some embodiments, a system for calibrating the position of a printer head comprises a means for calibrating the printer head along the x-axis, y-axis, and z-axis. In some embodiments, the means for calibrating a printer head along the x-axis, y-axis, and z-axis is laser alignment, optical alignment, mechanical alignment, piezoelectric alignment, magnetic alignment, electrical field or capacitance alignment, ultrasound alignment, or a combination thereof.

In some embodiments, a system for calibrating the position of a printer head comprises a means for calibrating the printer head along the x-axis, y-axis, and z-axis. In some embodiments, the means for calibrating a printer head along the x-axis, y-axis, and z-axis is laser alignment. In some embodiments, the laser alignment means comprises at least one laser. In some embodiments, the laser alignment means comprises a plurality of lasers.

In some embodiments, the laser alignment means it has any suitable accuracy. In various embodiments, suitable accuracies include those of about ±5, 10, 20, 30, 40, or 50 μm on any axis. In some embodiments, the laser alignment means is accurate to ±40 μm on the vertical axis and ±20 μm on the horizontal axis.

In some embodiments, the laser path is uninterrupted between the laser source and the measurement point. In some embodiments, the laser path is altered by up to 179° by use of a reflective surface or optical lens. In some embodiments, the laser path is altered by 90°. In some embodiments, a horizontal laser beam is used to measure in a vertical path by deflection using a reflective surface. In some embodiments, a vertical laser beam is used to measure in a horizontal path by deflection using a reflective surface.

### Bio-Ink

Disclosed herein are three-dimensional biological tubes and methods for the fabrication of bioprinted biological tubes that maintain cellular compartments post-fabrication without compromising cell viability and function. In some embodiments, cells are bioprinted by depositing or extruding bio-ink from a bioprinter. In some embodiments, “bio-ink” includes liquid, semi-solid, or solid compositions comprising a plurality of cells. In some embodiments, bio-ink comprises liquid or semi-solid cell solutions, cell suspensions, or cell concentrations. In some embodiments, a cell solution, suspension, or concentration comprises a liquid or semi-solid (e.g., viscous) carrier and a plurality of cells. In some embodiments, the carrier is a suitable cell nutrient media, such as those described herein.

In some embodiments, bio-ink comprises a plurality of cells that optionally cohere into multicellular aggregates prior to bioprinting. In some embodiments, bio-ink comprises a plurality of cells and is bioprinted to produce a specific planar and/or laminar geometry; wherein cohesion of the individual cells within the bio-ink takes place before, during, and/or after bioprinting. In some embodiments, the bio-ink is produced by collecting a plurality of cells in a fixed volume; wherein the cellular component(s) represent between about 30% and about 100%, about 30% and about 90%, about 30% and about 80%, about 30% and about 70%, about 30% and about 60%, about 30% and about 50%, about 30% and about 40%, about 40% and about 100%, about 40% and about 90%, about 40% and about 80%, about 40% and about 70%, about 40% and about 60%, about 40% and about 50%, about 50% and about 100%, about 50% and about 90%, about 50% and about 80%, about 50% and about 70%, about 50% and about 60%, about 60% and about 100%, about 60% and about 90%, about 60% and about 80%, about 60% and about 70%, about 70% and about 100%, about 70% and about 90%, about 70% and about 80%, about 80% and about 100%, about 80% and about 90%, or about 90% and about 100% of the total volume. In some embodiments, the bio-ink consists essentially of cells.

In some embodiments, bio-ink comprises semi-solid or solid multicellular aggregates or multicellular bodies. In further embodiments, the bio-ink is produced by 1) mixing a plurality of cells or cell aggregates and a biocompatible liquid or gel in a pre-determined ratio to result in bio-ink, and 2) compacting the bio-ink to produce the bio-ink with a desired cell density and viscosity. In some embodiments, the compacting of the bio-ink is achieved by centrifugation, tangential flow filtration (“TFF”), or a combination thereof. In some embodiments, the compacting of the bio-ink results in a composition that is extrudable, allowing formation of multicellular aggregates or multicellular bodies. In some embodiments, “extrudable” means able to be shaped by forcing (e.g., under pressure) through a nozzle or orifice (e.g., one or more holes or tubes). In some embodiments, the compacting of the bio-ink results from growing the cells to a suitable density. The cell density necessary for the bio-ink will vary with the cells being used and the biological tube being produced.

In some embodiments, the bio-ink is a viscous liquid, a semi-solid, or a solid. In some embodiments, the bio-ink is a viscous liquid. In some embodiments, the bio-ink is a semi-solid. In some embodiments, the bio-ink is a solid. In some embodiments, the viscosity of the bio-ink is between about 100 centipoise (cP) and about 200,000 cP, about 100 cP and about 100,000 cP, about 100 cP and about 50,000 cP, about 100 cP and about 20,000 cP, about 100 cP and about 10,000 cP, about 100 cP and about 5,000 cP, about 100 cP and about 2,000 cP, about 100 cP and about 1,000 cP, about 100 cP and about 500 cP, about 100 cP and about 200 cP, about 200 cP and about 200,000 cP, about 200 cP and about 100,000 cP, about 200 cP and about 50,000 cP, about 200 cP and about 20,000 cP, about 200 cP and about 10,000 cP, about 200 cP and about 5,000 cP, about 200 cP and about 2,000 cP, about 200 cP and about 1,000 cP, about 200 cP and about 500 cP, about 500 cP and about 200,000 cP, about 500 cP and about 100,000 cP, about 500 cP and about 50,000 cP, about 500 cP and about 20,000 cP, about 500 cP and about 10,000 cP, about 500 cP and about 5,000 cP, about 500 cP and about 2,000 cP, about 200 cP and about 1,000 cP, about 1,000 cP and about 200,000 cP, about 1,000 cP and about 100,000 cP, about 1,000 cP and about 50,000 cP, about 1,000 cP and about 20,000 cP, about 1,000 cP and about 10,000 cP, about 1,000 cP and about 5,000 cP, about 1,000 cP and about 2,000 cP, about 2,000 cP and about 200,000 cP, about 2,000 cP and about 100,000 cP, about 2,000 cP and about 50,000 cP, about 2,000 cP and about 20,000 cP, about 2,000 cP and about 10,000 cP, about 2,000 cP and about 5,000 cP, about 5,000 cP and about 200,000 cP, about 5,000 cP and about 100,000 cP, about 5,000 cP and about 50,000 cP, about 5,000 cP and about 20,000 cP, about 5,000 cP and about 10,000 cP, about 10,000 cP and about 200,000 cP, about 10,000 cP and about 100,000 cP, about 10,000 cP and about 50,000 cP, about 10,000 cP and about 20,000 cP, about 20,000 cP and about 200,000 cP, about 20,000 cP and about 100,000 cP, about 20,000 cP and about 50,000 cP, about 50,000 cP and about 200,000 cP, about 50,000 cP and about 100,000 cP, or about 100,000 cP and about 200,000 cP.

In some embodiments, the bio-ink is a filament. In some embodiments, the bio-ink is a continuous filament. In some embodiments, the bio-ink is a continuous filament with a length between about 30 mm and about 5,000 mm, about 30 mm and about 2,000 mm, about 30 mm and about 1,000 mm, about 30 mm and about 500 mm, about 30 mm and about 300 mm, about 30 mm and about 200 mm, about 30 mm and about 150 mm, about 30 mm and about 100 mm, about 30 mm and about 50 mm, about 50 mm and about 5,000 mm, about 50 mm and about 2,000 mm, about 50 mm and about 1,000 mm, about 50 mm and about 500 mm, about 50 mm and about 300 mm, about 50 mm and about 200 mm, about 50 mm and about 150 mm, about 50 mm and about 100 mm, about 100 mm and about 5,000 mm, about 100 mm and about 2,000 mm, about 100 mm and about 1,000 mm, about 100 mm and about 500 mm, about 100 mm and about 300 mm, about 100 mm and 200 mm, about 100 mm and about 150 mm, about 150 mm and about 5,000 mm, about 150 mm and about 2,000 mm, about 150 mm and about 1,000 mm, about 150 mm and about 500 mm, about 150 mm and about 300 mm, about 150 mm and about 200 mm, about 200 mm and about 5,000 mm, about 200 mm and about 2,000 mm, about 200 mm and about 1,000 mm, about 200 mm and about 500 mm, about 200 mm and about 300 mm, about 300 mm and about 5,000 mm, about 300 mm and about 2,000 mm, about 300 mm and about 1,000 mm, about 300 mm and about 500 mm, about 500 mm and about 5,000 mm, about 500 mm and about 2,000 mm, about 500 mm and about 1,000 mm, about 1,000 mm and about 5,000 mm, about 1,000 mm and about 2,000 mm, or between about 2,000 mm and about 5,000 mm.

In some embodiments, the bio-ink is a continuous filament with a diameter between about 10 μm and about 1000 μm, about 10 μm and about 500 μm, about 10 μm and about 250 μm, about 10 μm and about 200 μm, about 10 μm and about 100 μm, about 10 μm and about 50 μm, about 50 μm and about 1000 μm, about 50 μm and about 500 μm, about 50 μm and about 250 μm, about 50 μm and about 200 μm, about 50 μm and about 100 μm, about 100 μm and about 1000 μm, about 100 μm and about 500 μm, about 100 μm and about 250 μm, about 100 μm and about 200 μm, about 200 μm and about 1000 μm, about 200 μm and about 500 μm, about 200 μm and about 250 μm, about 250 μm and about 1000 μm, about 250 μm and about 500 μm, or between about 500 μm and about 1000 μm.

In some embodiments, the bio-ink comprises between about 1 million and about 1 billion, about 1 million and about 800 million, about 1 million and about 600 million, about 1 million and about 400 million, about 1 million and about 200 million, about 1 million and about 100 million, about 1 million and about 50 million, about 1 million and about 10 million, about 1 million and about 5 million, about 5 million and about 1 billion, about 5 million and about 800 million, about 5 million and about 600 million, about 5 million and about 400 million, about 5 million and about 200 million, about 5 million and about 100 million, about 5 million and about 50 million, about 5 million and about 10 million, 10 million and about 1 billion, about 10 million and about 800 million, about 10 million and about 600 million, about 10 million and about 400 million, about 10 million and about 200 million, about 10 million and about 100 million, about 10 million and about 50 million, about 50 million and about 1 billion, about 50 million and about 800 million, about 50 million and about 600 million, about 50 million and about 400 million, about 50 million and about 200 million, about 50 million and about 100 million, 100 million and about 1 billion, about 100 million and about 800 million, about 100 million and about 600 million, about 100 million and about 400 million, about 100 million and about 200 million, 200 million and about 1 billion, about 200 million and about 800 million, about 200 million and about 600 million, about 200 million and about 400 million, about 400 million and about 1 billion, about 400 million and about 800 million, about 400 million and about 600 million, about 600 million and about 1 billion, about 600 million and about 800 million, or about 800 million and about 1 billion cells per milliliter. In some embodiments, the bio-ink comprises between about 50 million and about 400 million cells per milliliter. In some embodiments, the bio-ink comprises between 200 million and 400 million cells per milliliter.

In some embodiments, the cells of the bio-ink are cohered and/or adhered. In some embodiments, “cohere,” “cohered,” and “cohesion” refer to cell-cell adhesion properties that bind cells, multicellular aggregates, multicellular bodies, and/or layers thereof. In some embodiments, the terms are used interchangeably with “fuse,” “fused,” and “fusion.” In some embodiments, the bio-ink additionally comprises support material, cell culture medium (or supplements thereof), extracellular matrix (or components thereof), cell adhesion agents, cell death inhibitors, anti-apoptotic agents, anti-oxidants, extrusion compounds, and combinations thereof.

In some embodiments, the cells are any suitable cell. In some embodiments, the cells are living cells. In some embodiments, the cells are vertebrate cells, mammalian cells, human cells, or combinations thereof. In some embodiments, the type of cell used in a method disclosed herein depends on the type of biological tube being produced. In some embodiments, the bio-ink comprises one type of cell (also referred to as a “homogeneous” or “monotypic” bio-ink). In some embodiments, the bio-ink comprises more than one Type of cell (also referred to as a “heterogeneous” or “polytypic” bio-ink).

cell culture media

in some embodiments, the bio-ink comprises a cell culture medium. The cell Culture medium is any suitable medium. In some embodiments, suitable cell culture Media is Dulbecco's Phosphate Buffered Saline, Earle'S Balanced Salts, Hanks' Balanced Salts, Tyrode's Salts, Alsever's Solution, Gey's Balanced Salt Solution, Kreb's-Henseleit Buffer Modified, Kreb's-Ringer Bicarbonate Buffer, Puck's Saline, Dulbecco's Modified Eagle's Medium, Dulbecco's Modified Eagle's Medium/Nutrient f-12 Ham, Nutrient Mixture F-10 Ham (Ham's F-10), Medium 199, Minimum Essential Medium Eagle, RPMI-1640 Medium, Ames' Media, Bgjb Medium (Fitton-Jackson Modification), Click's Medium, CMRL-1066 Medium, Fischer's Medium, Glascow Minimum Essential Medium (Gmem), Iscove's Modified Dulbecco's Medium (Imdm), L-15 Medium (Leibovitz), Mccoy's 5a Modified Medium, Nctc Medium, Swim's S-77 Medium, Waymouth medium, William's Medium E, or combinations thereof. In some Embodiments, the cell culture medium is modified or supplemented. In some Embodiments, the cell culture medium further comprises albumin, selenium, transferrins, Fetuins, sugars, amino acids, vitamins, growth factors, cytokines, hormones, antibiotics, lipids, lipid carriers, cyclodextrins, platelet-rich plasma, or a combination thereof.

### Extracellular Matrix

In some embodiments, the bio-ink further comprises one or more components of an extracellular matrix (ECM) or derivatives thereof. In some embodiments, “extracellular matrix” includes proteins that are produced by cells and transported out of the cells into the extracellular space, where they serve as a support to hold tissues together, to provide tensile strength, and/or to facilitate cell signaling. Examples of extracellular matrix components include, but are not limited to, collagens, fibronectin, laminins, hyaluronates, elastin, and proteoglycans. In some embodiments, the bio-ink comprises various ECM proteins (e.g., gelatin, fibrinogen, fibrin, collagens, fibronectin, laminins, elastin, and/or proteoglycans). The ECM components or derivatives of ECM components are optionally added to the cell paste used to form the bio-ink. The ECM components or derivatives of ECM components added to the bio-ink are optionally purified from a human or animal source, or produced by recombinant methods known in the art. Alternatively, the ECM components or derivatives of ECM components are naturally secreted by the cells in the elongate cellular body, or the cells used to make the elongate cellular body are optionally genetically manipulated by any suitable method known in the art to vary the expression level of one or more ECM components or derivatives of ECM components and/or one or more cell adhesion molecules or cell-substrate adhesion molecules (e.g., selectins, integrins, immunoglobulins, and adherins). In some embodiments, the ECM components or derivatives of ECM components promote cohesion of the cells in the bio-inks. For example, gelatin and/or fibrinogen is suitably added to the cells, which is used to form the bio-ink. The fibrinogen is converted to fibrin by the addition of thrombin.

In some embodiments, the bio-ink further comprises an agent that inhibits cell death (e.g., necrosis, apoptosis, or autophagocytosis). In some embodiments, the bio-ink further comprises an anti-apoptotic agent. Agents that inhibit cell death include, but are not limited to, small molecules, antibodies, peptides, peptibodies, or combination thereof. In some embodiments, the agent that inhibits cell death is selected from: anti-TNF agents, agents that inhibit the activity of an interleukin, agents that inhibit the activity of an interferon, agents that inhibit the activity of an GCSF (granulocyte colony-stimulating factor), agents that inhibit the activity of a macrophage inflammatory protein, agents that inhibit the activity of TGF-B (transforming growth factor B), agents that inhibit the activity of an MMP (matrix metalloproteinase), agents that inhibit the activity of a caspase, agents that inhibit the activity of the MAPK/JNK signaling cascade, agents that inhibit the activity of a Src kinase, agents that inhibit the activity of a JAK (Janus kinase), or a combination thereof. In some embodiments, the bio-ink comprises an anti-oxidant. In some embodiments, the bio-ink comprises oxygen-carriers or other cell-specific nutrients.

### Cell Carrier Material

In some embodiments, the bio-ink further comprises a cell carrier material or an extrusion compound (i.e., a compound that modifies the extrusion properties of the bio-ink). Examples of cell carrier materials include, but are not limited to gels, hydrogels, peptide hydrogels, amino acid-based gels, surfactant polyols (e.g., Pluronic F-127 or PF-127), thermo-responsive polymers, hyaluronates, alginates, extracellular matrix components (and derivatives thereof), collagens, gelatin, other biocompatible natural or synthetic polymers, nanofibers, and self-assembling nanofibers. In some embodiments, the cell carrier material is removed after bioprinting by physical, chemical, or enzymatic means.

In some embodiments, the cell carrier material is a hydrogel. In certain embodiments, the biological tubes fabricated by the methods of this disclosure utilize at least one hydrogel such as collagen, hyaluronate, hyaluronan, fibrin, alginate, agarose, chitosan, chitin, cellulose, pectin, starch, polysaccharides, fibrinogen/thrombin, fibrillin, elastin, gum, cellulose, agar, gluten, casein, albumin, vitronectin, tenascin, entactin/nidogen, glycoproteins, glycosaminoglycans (GAGs), and proteoglycans which may contain for example chrondroitin sulfate, fibronectin, keratin sulfate, laminin, heparan sulfate proteoglycan, decorin, aggrecan, perlecan, and combinations thereof. In some embodiments, suitable hydrogels are synthetic polymers. In some embodiments, the hydrogel is derived from poly(acrylic acid) and derivatives thereof, poly(ethylene oxide) and copolymers thereof, poly(vinyl alcohol), polyphosphazene, and combinations thereof. In some embodiments, the hydrogel is NOVOGEL®, agarose, alginate, gelatin, MATRIGEL®, hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, or combinations thereof. In some embodiments, the cell carrier material is NOVOGEL®. In some embodiments, the cell carrier material is collagen hydrogel. In some embodiments, the cell carrier material is hyaluronic acid hydrogel. In some embodiments, the cell carrier material is a mixture of hyaluronic acid and gelatin. In some embodiments, the cell carrier material is gelatin.

### Cell Types

In some embodiments, any vertebrate cell is suitable for inclusion in bio-ink and the three-dimensional biological tube. In some embodiments, the cells are living cells. In some embodiments, the cells are contractile or muscle cells (e.g., skeletal muscle cells, cardiomyocytes, smooth muscle cells, and myoblasts), connective tissue cells (e.g., bone cells, cartilage cells, fibroblasts, and cells differentiating into bone forming cells, chondrocytes, or lymph tissues), bone marrow cells, endothelial cells, skin cells, epithelial cells, breast cells, vascular cells, blood cells, lymph cells, neural cells, Schwann cells, gut cells, gastrointestinal cells, liver cells, pancreatic cells, lung cells, tracheal cells, corneal cells, genitourinary cells, kidney cells, reproductive cells, adipose cells, parenchymal cells, pericytes, mesothelial cells, stromal cells, undifferentiated cells (e.g., embryonic cells, stem cells, and progenitor cells, adult stem cells, induced pluripotent stem cells (iPS cells), cancer stem cells), endoderm-derived cells, mesoderm-derived cells, ectoderm-derived cells, cells expressing disease associated antigen or associated with disease (e.g., cancer), and combinations thereof. In some embodiments, the bio-ink comprises fibroblasts. In some embodiments, the bio-ink comprises fibroblasts of dermal origin. In some embodiments, the bio-ink comprises fibroblasts of renal origin. In some embodiments, the bio-ink comprises fibroblasts of vascular origin. In some embodiments, the bio-ink comprises endothelial cells. In some embodiments, the bio-ink comprises fibroblasts and endothelial cells. In some embodiments, the bio-ink comprises keratinocytes. In some embodiments, the bio-ink comprises melanocytes. In some embodiments, the bio-ink comprises hepatocytes. In some embodiments, the bio-ink comprises stellate cells. In some embodiments, the bio-ink comprises epidermal cells. In some embodiments, the bio-ink comprises dermal cells. In some embodiments, the bio-ink comprises epithelial cells. In some embodiments, the bio-ink comprises renal tubular epithelial cells. In some embodiments, the bio-ink consists essentially of a single cell type. In some embodiments, the bio-ink consists essentially of two cell types. In some embodiments, the bio-ink consists essentially of three cell types. In some embodiments, the bio-ink consists essentially of four cell types. In some embodiments, the bio-ink consists essentially of human cells. In some embodiments, the bio-ink consists essentially of human primary cells.

In some embodiments, the cells have been modified biologically, chemically, or physically. Biological modifications include genetic modifications such as transfection, transduction, or infection with a transgene that encodes wild-type, dominant negative, truncated, or mutant protein. The transgene can also encode an miRNA, siRNA, shRNA, or an antisense RNA. The transgene can be maintained transiently or stably integrated into the cellular genome. Transfection can be achieved by cationic lipids, calcium phosphate, and electroporation, or through uptake of DNA without a specific transfection means. The cells can be virally transduced with any viral vector commonly used for these purposes such as a retrovirus, lentivirus, adenovirus, adeno associated virus, or vaccinia virus. The modification can be chemical such as treatment with a mutagen, antibiotic, antifungal, antiviral, HDAC inhibitor, chemotherapeutic, fluorescent labeling, tracking dye, or cell permanent or cell impermanent dyes. The modifications can be physical such as radiation, electromagnetic radiation, X-rays, or hot and cold shocks.

In some embodiments, the cells are adult, differentiated cells. In some embodiments, “differentiated cells” are cells with a tissue-specific phenotype consistent with, for example, a muscle cell, a fibroblast, or an endothelial cell at the time of isolation, wherein tissue-specific phenotype (or the potential to display the phenotype) is maintained from the time of isolation to the time of use. In other embodiments, the cells are adult, non-differentiated cells. In further embodiments, “non-differentiated cells” are cells that do not have, or have lost, the definitive tissue-specific traits of for example, muscle cells, fibroblasts, or endothelial cells. In some embodiments, non-differentiated cells include stem cells. In further embodiments, “stem cells” are cells that exhibit potency and self-renewal. Stem cells include, but are not limited to, totipotent cells, pluripotent cells, multipotent cells, oligopotent cells, unipotent cells, and progenitor cells. In various embodiments, stem cells are embryonic stem cells, adult stem cells, amniotic stem cells, and induced pluripotent stem cells. In other embodiments, the cells are a mixture of adult, differentiated cells and adult, non-differentiated cells.

### Pre-Formed Scaffold

In some embodiments, disclosed herein are engineered biological tubes that are free or substantially free of any pre-formed scaffold. In some embodiments, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and/or organ and not removed from the tissue and/or organ. In still further embodiments, decellularized tissue scaffolds include decellularized native tissues or decellularized cellular material generated by cultured cells in any manner; for example, cell layers that are allowed to die or are decellularized, leaving behind the ECM they produced while living.

In some embodiments, the engineered biological tubes do not utilize any pre-formed scaffold, e.g., for the formation of the tube, any layer of the tube, or formation of the tube's shape. As a non-limiting example, the engineered biological tubes of the present invention do not utilize any pre-formed, synthetic scaffolds such as polymer scaffolds, pre-formed extracellular matrix layers, or any other type of pre-formed scaffold at the time of manufacture or at the time of use. In some embodiments, the engineered tissues are substantially free of any pre-formed scaffolds. In further embodiments, the cellular components of the tissues contain a detectable, but trace or trivial amount of scaffold, e.g., less than 2.0%, less than 1.0%, or less than 0.5% of the total composition. In some embodiments, trace or trivial amounts of scaffold are insufficient to affect long-term behavior of the biological tube or interfere with its primary biological function. In some embodiments, scaffold components are removed post-printing, by physical, chemical, or enzymatic methods, yielding an engineered biological tube that is free or substantially-free of scaffold components.

### Cross-Linkers

In some embodiments, the method of preparing biological tubes does not require any cross-linking. In some embodiments, the method of preparing biological tubes does not require chemical cross-linking (i.e, using gluteraldehyde or bis-epoxide); cross-linking by ionic cross-linkers; cross-linking by any cationic or anionic cross-linkers; cross-linking by calcium, magnesium, sodium, chloride, alginate, or any combination thereof; cross-linking by enzymatic cross-linkers; physical cross-linking; photo cross-linking; or radiation cross-linking, including ultraviolet or visible light.

### Biological Tubes

Described herein are methods of rapidly fabricating biological tubes. The methodologies described herein allow for the high-throughput generation of extra-long tubes including 30 mm long tubes printed in 3.5 minutes as compared to 35-60 minutes per tube achieved with existing methods. In some embodiments, solid or semi-solid aggregated cell paste (e.g., a cell/hydrogel mixture) or bio-ink (e.g., a 100% cellular sample) is printed onto the surface of the rotating mandrel using continuous deposition or capillary deposition. See, e.g., Examples 2, 3, and 4.

In some embodiments, a continuous deposition mode is used and a tubular construct is created through extrusion of solid or semi-solid cellular cell mixture of, for example, 250-300 million cells/mL and a hydrogel such hyaluronic acid (HA). Other suitable hydrogels, functional hydrogels, and additives, such as collagen are optionally used. In some embodiments, a capillary deposition mode is used, wherein solid or semi-solid bio-ink absent of exogenous biomaterial is hand-wrapped around a mandrel.

In some embodiments, the methods comprise preparing one or more bio-inks comprising living cells. In some embodiments, the bio-ink comprises a solid or semi-solid bio-ink filament. In some embodiments, the methods comprise depositing the bio-ink filament onto a rotating biocompatible mandrel. In some embodiments, the mandrel optionally comprises a removable form-fitting sheath to receive the bio-ink. In some embodiments, the methods comprise maturing the deposited bio-ink filament while on the mandrel in a cell culture media. In some embodiments, the deposited bio-ink is maintained in culture on the mandrel for about 4 hours to about 12 hours. In some embodiments, the methods comprise removing the biological tube from the mandrel or sheath. This step is optionally achieved by using forceps or by dissolving or digesting the mandrel or sheath. In some embodiments, the methods comprise maturing the biological tube in a cell culture media after it has been removed from the mandrel. In some embodiments, the biological tube is maintained in culture for about 8 hours to about 2 weeks.

In some embodiments, the methodologies described herein produce uniform tubes. In some embodiments, the biological tube has a uniform outer surface. In some embodiments, the biological tube has a uniform inner surface. In some embodiments, the biological tube has a uniform wall thickness. By the term “uniform” is intended that the particular dimension does not vary by more than 10% along the length of the tube.

The methodologies described herein are optionally used to create multilaminate or multilayered tubes comprising distinct compartments of cells (e.g., smooth muscle cells, fibroblasts, and endothelial cells, etc.) mimicking aspects of the native architecture of blood vessels—particularly in the formation of an endothelial cell-only layer within the lumen of the construct. The biological tubes described herein suitably comprise 1, 2, 3, 4, 5, 6, 7, 8, or more layers. Layers are optionally applied by a spray deposition technique, such as ink-jet bioprinting. Layers are also optionally applied coiling or winding a bio-ink filament around a rotating mandrel as described herein. In some embodiments, the biological tube is a branched tube.

In some embodiments, the biological construct comprises two or more layers. In some embodiments, a biological construct comprising at least two layers comprises at least one layer prepared using living cells and at least one layer prepared without living cells. In some embodiments, the at least one layer prepared without living comprises a gel, hydrogel, peptide hydrogel, amino acid-based gel, surfactant polyol (e.g., Pluronic F-127 or PF-127), thermo-responsive polymer, hyaluronate, alginate, extracellular matrix component (and derivatives thereof), collagen, gelatin, other biocompatible natural or synthetic polymer, nanofiber, self-assembling nanofiber, or combinations thereof. In some embodiments, the at least one layer prepared without living cells comprises a hyaluronic acid hydrogel, collagen, gelatin, alginate, or combinations thereof.

In some embodiments, the at least one layer prepared without living cells comprises (by volume) between about 0.5% and about 20%, about 0.5% and about 10%, about 0.5% and about 5%, about 0.5% and about 1%, about 1% and about 20%, about 1% and about 10%, about 1% and about 5%, about 5% and about 20%, about 0.5% and about 10%, or about 10% and about 20% hyaluronic acid hydrogel. In some embodiments, the at least one layer prepared without living cells comprises (by volume) between about 0.5% and about 20%, about 0.5% and about 10%, about 0.5% and about 5%, about 0.5% and about 1%, about 1% and about 20%, about 1% and about 10%, about 1% and about 5%, about 5% and about 20%, about 5% and about 10%, or about 10% and about 20% collagen. In some embodiments, the at least one layer prepared without living cells comprises (by volume) between about 0.5% and about 20%, about 0.5% and about 10%, about 0.5% and about 5%, about 0.5% and about 1%, about 1% and about 20%, about 1% and about 10%, about 1% and about 5%, about 5% and about 20%, about 5% and about 10%, or about 10% and about 20% alginate. In some embodiments, the at least one layer prepared without living cells comprises (by volume) between about 0.5% and about 20%, about 0.5% and about 10%, about 0.5% and about 5%, about 0.5% and about 1%, about 1% and about 20%, about 1% and about 10%, about 1% and about 5%, about 5% and about 20%, about 5% and about 10%, or about 10% and about 20% gelatin.

In some embodiments, tubes, of various lengths, diameters, and branched architectures are produced. In some embodiments, the biological tube has a lumen about 250 μm to about 10 cm in diameter. In some embodiments, the biological tube is an engineered multilayered vascular tube and at least one layer was prepared using a bio-ink filament comprising living vascular cells. In some embodiments, the engineered multilayered vascular tube has a lumen about 250 μm to about 25 mm in diameter. In some embodiments, the biological tube is an engineered multilayered pulmonary tube and at least one layer was prepared using a bio-ink filament comprising living pulmonary cells. In some embodiments, the engineered multilayered pulmonary tube has a lumen about 250 μm to about 30 mm in diameter. In some embodiments, the biological tube is an engineered multilayered intestinal tube and at least one layer was prepared using a bio-ink filament comprising living intestinal cells. In some embodiments, the engineered multilayered intestinal tube has a lumen 2 cm to 5 cm in diameter (e.g., roughly the diameter of an adult human small intestine). In some embodiments, the intestinal tube has a lumen 8 cm to 12 cm in diameter (e.g., roughly the diameter of an adult human large intestine). In some embodiments, the three-dimensional, engineered, biological constructs described herein include one or more cellular layers. In further embodiments, the layers are stratified. In some embodiments, the engineered biological constructs include a basal layer. In some embodiments, the biological constructs described herein are skin tissues. In some embodiments, the biological constructs described herein are kidney tissues. In some embodiments, the biological constructs described herein are liver tissues. In some embodiments, the biological constructs described herein are lung tissues. In some embodiments, the biological constructs described herein are gut tissues. In some embodiments, the biological constructs described herein are intestinal tissues.

Differences from Previous Engineered Tubes and Native Tubes

In some embodiments, the three-dimensional, engineered biological constructs described herein are distinguished from native (e.g., non-engineered) constructs by virtue of the fact that they are non-innervated (e.g., substantially free of nervous tissue), substantially free of mature vasculature, and/or substantially free of blood components. For example, in various embodiments, the three-dimensional, engineered biological constructs are free of plasma, red blood cells, platelets, and the like and/or endogenously-generated plasma, red blood cells, platelets, and the like. In certain embodiments, the biological constructs lack hemoglobin. In some embodiments, the biological constructs lack innervation or neurons. In some embodiments, the biological constructs lack neuronal markers such as any of: Beat III tubulin, MAP2, NeuN, and neuron specific enolase. In some embodiments, the engineered biological constructs are species chimeras, wherein at least one cell or cell-type of the tissue is from a different mammalian species then another cell or cell-type of the tissue. In some embodiments, the biological constructs described herein are marked by an increased basal metabolic rate then tissue in vivo or ex vivo. In some embodiments, the biological constructs described herein are marked by an increased proliferative rate then tissue in vivo or in ex vivo culture. In some embodiments, the biolocial constructs described herein are marked by an increased cell size when compared to cells in tissue in vivo or in ex vivo culture.

In some embodiments, one or more components of the biological tubes described herein are bioprinted, which comprises an additive fabrication process. In some embodiments, the biological tubes lack a synthetic or biological scaffold. Substantial elimination of scaffolds represents an improvement over prior methodologies by reducing immunogenicity and improving authenticity of the resulting constructs.

In some embodiments, through the methods of fabrication, the fabricator exerts significant control over the composition of the resulting biological tubes described herein. As such, the engineered biological tubes described herein optionally comprise any of the layers, structures, compartments, and/or cells of native tissue. Conversely, the engineered biological tubes described herein optionally lack any of the layers, structures, compartments, and/or cells of native tissue. By way of example, in some embodiments, the biological tubes lack at least one of innervation, lymphatic tissue, perfusable supporting vasculature (e.g., mature vascular networks), and/or red blood cells.

In some embodiments, the three-dimensional, engineered biological are distinguished from tissues fabricated by prior technologies by virtue of the fact that they are three-dimensional, free of pre-formed scaffolds, consist essentially of cells, and have a high cell density. In some embodiments, the percentage of cells (volume/volume) in the engineered biological construct is between about 10% and about 100%, 10% and about 95%, about 10% and about 90%, about 10% and about 80%, about 10% and about 50%, about 10% and about 30%, about 30% and about 100%, about 30% and about 95%, about 30% and about 90%, about 30% and about 80%, about 30% and about 50%, about 50% and about 100%, about 50% and about 95%, about 50% and about 90%, about 50% and about 80%, about 80% and about 100%, about 80% and about 95%, about 80% and about 90%, about 90% and about 100%, about 90% and about 95%, or about 95% and 100%. In some embodiments, the engineered biological constructs have been exposed to incubations at different non-physiological temperatures at various times. For mammalian cells physiological temperature is defined as the normal body temperature of about 37° C.

### EXAMPLES

The following examples are illustrative and non-limiting, of the products and methods described herein. Suitable modifications and adaptations of the variety of conditions, formulations, and other parameters normally encountered in the field and which are obvious to those skilled in the art in view of this disclosure are within the spirit and scope of the invention.

### Example 1—Cell Aggregate (Bio-Ink) Preparation

Chinese Hamster Ovary (CHO) cells, transfected with N-cadherin (courtesy of A. Bershadsky, Weizmann Institute, Rehovot, Israel), can be infected with histone binding H2B-YFP retrovirus (provided by R. D. Lansford, Beckman Institute at California Institute of Technology). Confluent cell cultures (3-4×10⁶ cells/75 cm² TC dish) grown in Dulbecco's Modified Eagle Medium (DMEM, Gibco BRL Grand Island, N.Y.); supplemented with 10% FES (US Biotechnologies, Parkerford, Pa.), 10 μg/mL of penicillin, streptomycin, gentamicin, kanamycin, 400 μg/mL geneticin), can be washed twice with Hanks' Balanced Salt Solution (HBSS) containing 2 mM CaCl₂, then treated for 10 minutes with trypsin 0.1% (diluted from 2.5% stock, Gibco BRL, Grand Island, N.Y.). Depleted cells can be centrifuged at 2500 RPM for 4 minutes. The resulting pellet can be transferred into capillary micropipettes of 500 μm diameter and incubated at 37° C. with 5% CO₂ for 10 minutes.

The resulting pellet can be transferred into porous tubing (e.g., dialysis tubing, dialysis fiber, or similarly structured tubes with high porosity and pore sizes of 0.5-5 μm, and wall thicknesses of 50 μm to 500 μm) and then submerged in culture media and incubated at 37° C. with 5% CO₂ for 10 minutes to 12 hours prior to extrusion. The resulting pellet can be extruded through a coaxial nozzle as the center stream with a sheath of hydrogel material such as alginate (other suitable materials include, but are not limited to, collagen, hyaluronic acid, chitosan, or any other synthetic or naturally-occurring materials or derivative of synthetic or natural materials amenable to extrusion processes and subsequent digestion/removal), incubated in cell culture media at 37° C. with 5% CO₂ for 10 minutes to 12 hours, and subsequently removed via enzymatic digestion, thermal degradation, or other physical process to reveal a firm cylinder of bio-ink. The firm cylinders of cells can be removed from the pipettes, porous tubing, or derived from coaxial extrusion processes and can be utilized for tube forming processes described elsewhere in this document.

These techniques can be applied to single identity cell populations as well admixtures of cells including, but not limited to the cells listed below: HUVEC (Human Umbilical Vein Endothelial Cells), NHLF (Normal Human Lung Fibroblasts), BSMC (Bronchial Smooth Muscle Cells), HPAEC (Human Pulmonary Artery Endothelial Cells), RFP-HPAEC (RFP-Human Pulmonary Artery Endothelial Cells), Stellate (Human Hepatic Stellate Cells), Rooster MSC (Mesenchymal Cells), bmMSC (bone-marrow Mesenchymal Cells), HDF (Human Dermal Fibroblasts), GFP-HHDP (GFP-Human Hair Dermal Papilla), HHDP (Human Hair Dermal Papilla), LSEC (Liver Sinusoidal Endothelial Cells), RF (adult) (Renal Fibroblasts), Hep-G2 (Human liver carcinoma cell line), and NIH/3T3 (Mouse embryo fibroblast cell line).

### Example 2—Test of NOVOGEL® 3.0 as a Cell Carrier Material During the Winding Process

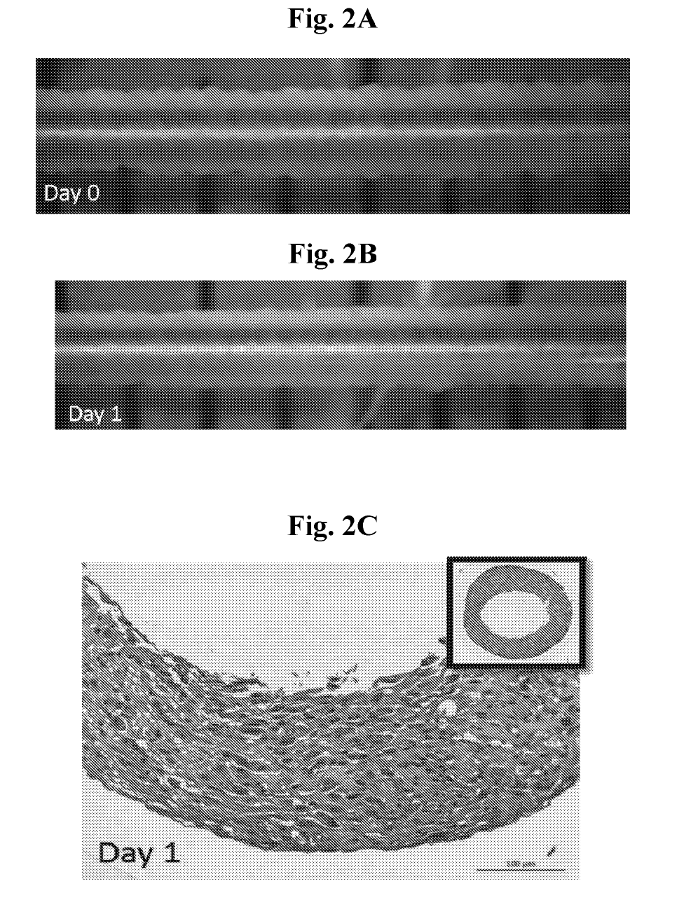

Tubular constructs consisting of NOVOGEL® 3.0 (Organovo, Inc., San Diego, Calif.) and 250 million cells/mL (50/50 mix of NHLFs and BSMCs) formed after overnight static conditioning (see FIGS. 2A and 2B). Print parameters can vary depending on the rate of tube deposition required, the size of the extrusion needle, and the size of the mandrel. Maximum values for these parameters across all needles and syringes are: Translation: 0.83 mm/s, Rotation: 100 rpm, and Extrusion: 20 mm/s or 33.38 μL/s.

The histological results indicate that some cell-cell interaction and attachment occurred, however, there were areas of cell encapsulation and immobilization. In addition, there was uneven distribution of the cells creating pockets within alginate that limited the construct from becoming fully cellular. See FIG. 2C.

### Example 3—Test of Collagen Hydrogel as Cell Carrier Material During the Winding Process

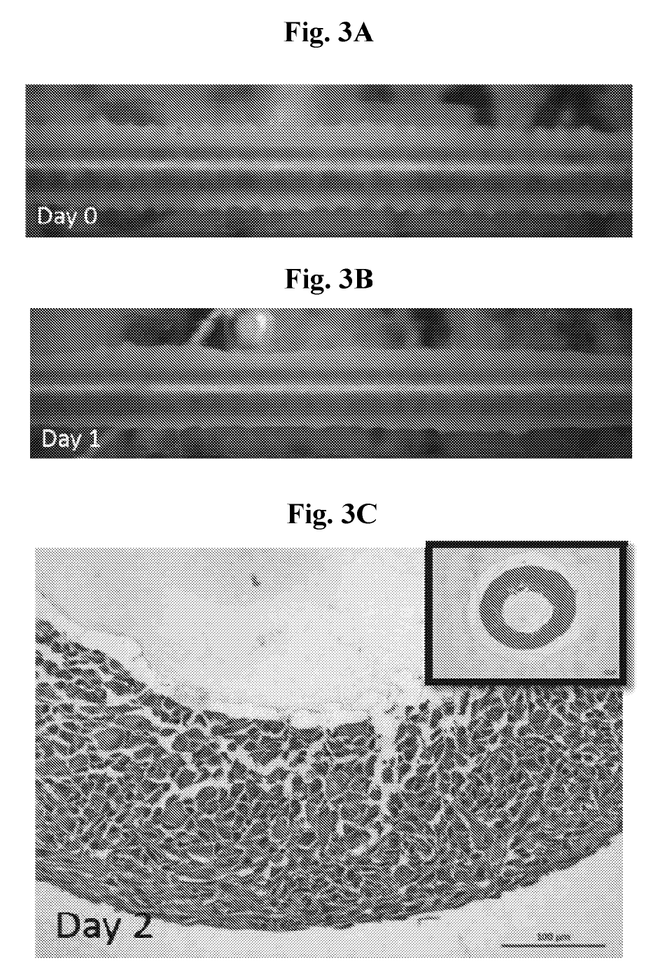

The collagen hydrogel with 200 million cells/mL (50/50 mix of NHLFs and BSMCs) also fused to form a tube around the mandrel (see FIGS. 3A and 3B). Print parameters can vary depending on the rate of tube deposition required, the size of the extrusion needle, and the size of the mandrel. Maximum values for these parameters across all needles and syringes are: Translation: 0.83 mm/s, Rotation: 100 rpm, and Extrusion: 20 mm/s or 33.38 μL/s.

The histological results from the constructs demonstrated minimal cell encapsulation with good cellularity and amorphous organization in the central regions of the tube wall with flat morphologies found on the abluminal and intimal surfaces of the tube. See FIG. 3C.

### Example 4—Test of HA Hydrogel for Cell Carrier Hydrogel During the Winding Process

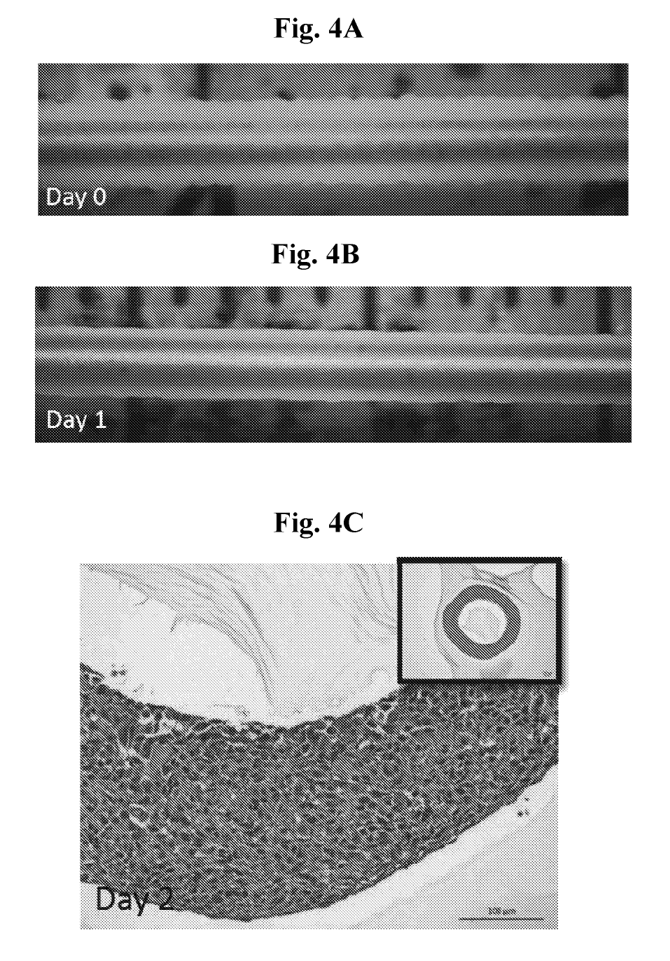

The hyaluronic acid (HA) hydrogel was printed with 200 million cells/mL (50/50 mix of NHLFs and BSMCs) resulted in tight coils that fused over time and contracted around the mandrel to form a complete tube (see FIGS. 4A and 4B). Print parameters can vary depending on the rate of tube deposition required, the size of the extrusion needle, and the size of the mandrel. Maximum values for these parameters across all needles and syringes are: Translation: 0.83 mm/s, Rotation: 100 rpm, and Extrusion: 20 mm/s or 33.38 μL/s.

The histological results suggest the tube was fully cellular and viable with uniform walls and an open lumen. See FIG. 4C.

### Example 5—Optimization of Cell Concentration and Hydrogel Composition

A study was performed to determine the optimal hydrogel and cell density based on the methodologies described in Examples 3 and 4. Four different groups were compared: (1) HA at 200 million cells/mL; (2) HA at 300 million cells/mL; (3) collagen at 200 million cells/mL; and (4) collagen at 300 million cells/mL.

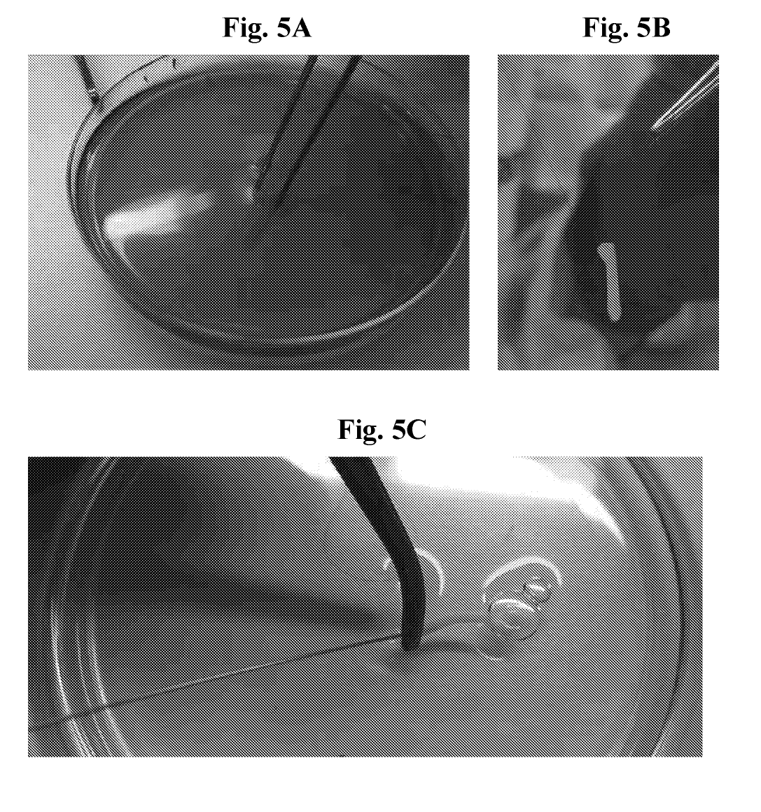

The response optimizer indicated HA tubes with a cell density of 300 million cells/mL exhibited the most desirable characteristics. The histological analysis of the collagen and HA tubular constructs suggests fully cellular, viable tubes with uniform walls and an open lumen. The tubes were easy to handle (see FIG. 5A), held a suture (see FIG. 5B), allowed perfusion of air through the lumen (see FIG. 5C), and had the highest suture using the suture pull-out test described in Trowbridge, E. A., et al., “Pericardial heterografts: a comparative study of suture pull-out and tissue strength,” J. Biomed. Eng. 11:311-314 (1989).

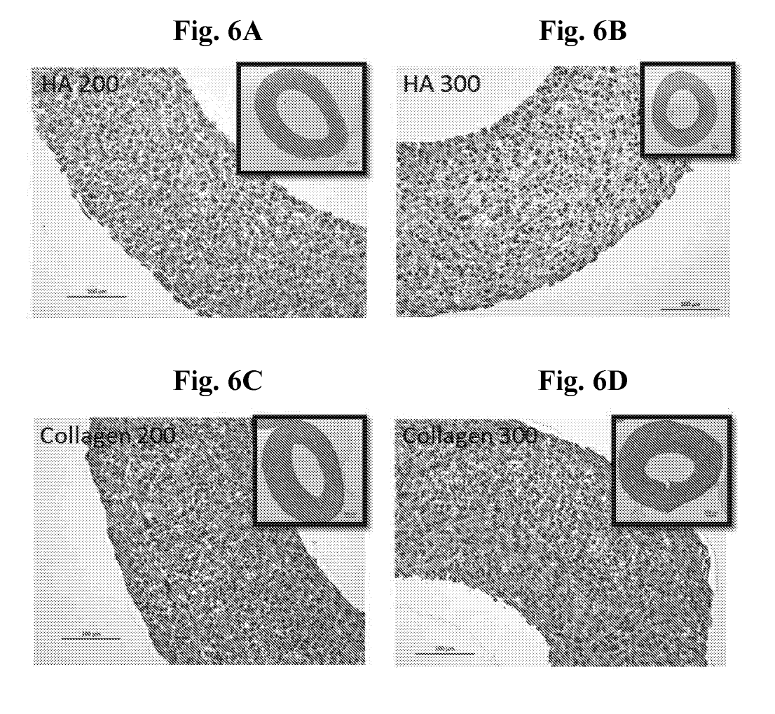

The histological results from the study suggest the tubes were viable with uniform walls and open lumens (see FIGS. 6A-6D). A difference between the cell densities was not noticeable though the collagen tubes appeared to have a higher cellularity.

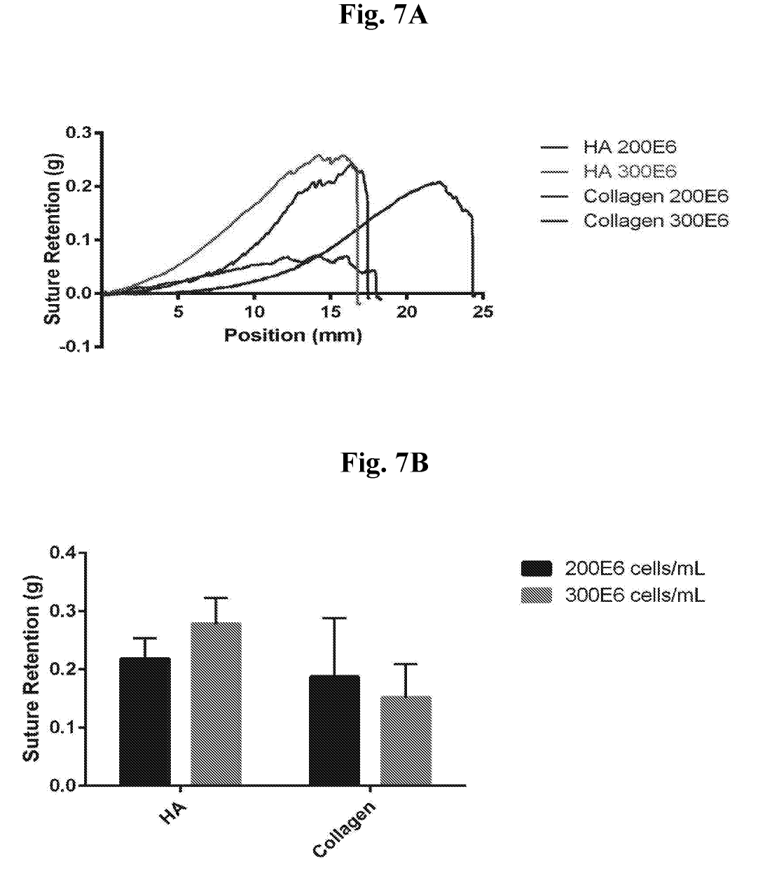

Lastly, the HA constructs had a higher suture retention strength than the collagen constructs (see FIGS. 7A and 7B). FIG. 7A illustrates gram-weights at which a single suture is pulled through the wall of a mandrel-generated tube. FIG. 7B illustrates that statistical analysis did not find a significant difference in the suture pull-out results at different cell concentrations.

### Example 6—Bi-Layered Constructs Formed with HPAECs as the Interior Layer

An inner 250 μm layer of 100% HPAECs in 6% gelatin (200×10⁶ cells/mL) was printed on the mandrel and covered by a 500 μm layer of 50% NHLF and 50% BSMC (300×10⁶ cells/mL) in a 50% HA and 50% gelatin solution. Print parameters for the respective layers are presented in Table 3 below.

TABLE 3

| | MANDREL | NEEDLE | |
|---|---|---|---|
| | ROTATION | TRANSLATION | PUMP EXTRUSION |
| LAYER | SPEED (rpm) | SPEED (mm/s) | RATE (μL/s) |
| INNER | 17 | 0.07 | 0.19 |
| OUTER | 17 | 0.14 | 0.35 |

The constructs were incubated statically at 30° C. overnight and then transferred to rotation at 37° C. for 5 days. All of the printed constructs remained attached to the mandrel up to day 6 post printing. After this incubation, the tubes were gently removed from the mandrel, fixed in 2% PFA and sent to histology for processing.

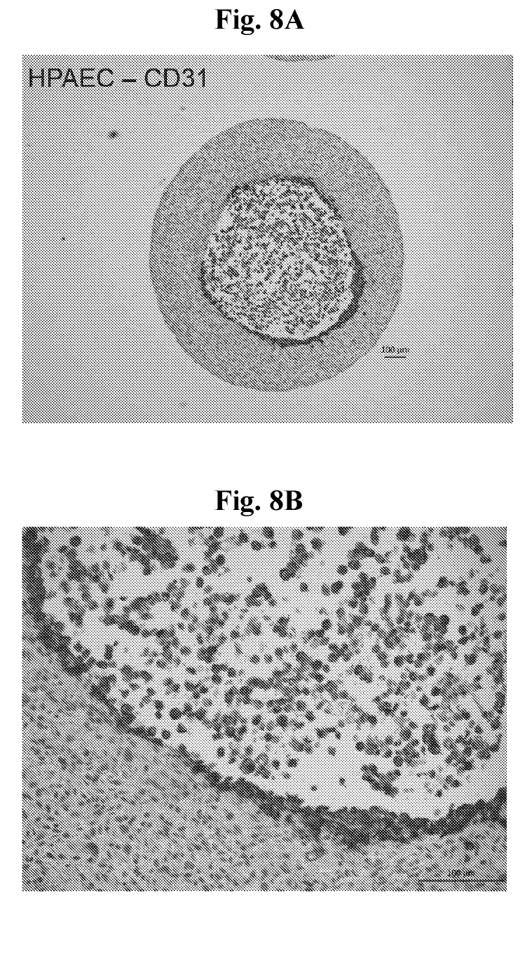

The histological results of the bi-layered biological construct stained for CD31 in the lumen of the tube show two discrete layers. See FIGS. 8A-8B.

### Example 7—Bi-Layered Constructs Formed with HUVECs as the Interior Layer

An inner 250 μm layer of 100% HUVEC cells in 6% gelatin (200×10⁶ cells/mL) was printed on the mandrel and covered by a 500 μm layer of 50% NHLF and 50% BSMC (300×10⁶ cells/mL) in 50% HA and 50% gelatin solution. Print parameters for the respective layers are presented in Table 4 below.

TABLE 4

| | MANDREL | NEEDLE | |
|---|---|---|---|
| | ROTATION | TRANSLATION | PUMP EXTRUSION |
| LAYER | SPEED (rpm) | SPEED (mm/s) | RATE (μL/s) |
| INNER | 17 | 0.07 | 0.19 |
| OUTER | 17 | 0.14 | 0.35 |

The constructs were incubated statically at 30° C. overnight and then transferred to rotation at 37° C. for 5 days. All of the printed constructs remained attached to the mandrel up to day 6 post printing. After this incubation, the tubes were gently removed from the mandrel, fixed in 2% PFA and sent to histology for processing.

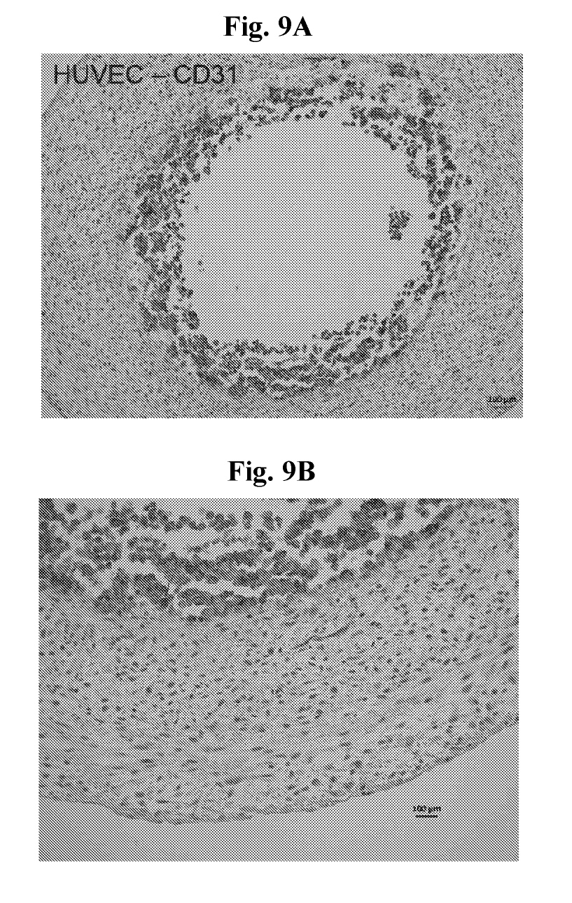

The histological results of the bi-layered biological construct stained for CD31 in the lumen of the tube show two discrete layers. See FIGS. 9A-9B.

### Example 8—Process for Preparing Bio-Ink

Intestinal myofibroblasts (IMF) (Lonza, Basel, Switzerland), mesenchymal stem cells (MSC) (RoosterBio, Inc., Frederick, Md.), and Caco-2 cells (Sigma-Aldrich, St. Louis, Mo.) were grown according to the manufacturer's instructions in Smooth Muscle Cell Growth Medium-2 (SMGM-2) (Lonza, Basel, Switzerland), hMSC High Performance Media (RoosterBio, Inc., Frederick, Md.), and Dulbecco's Modified Eagle Medium (DMEM) (Gibco Laboratories, Gaithersburg, Md.) supplemented with 15% FBS, 1× antibiotic antimycotic, 1×L-glutamine, respectively. Cells were harvested with 0.1% trypsin or TrypLE for 5-7 minutes following 2 washes with PBS. Cells were centrifuged at 200×g for 5 minutes. IMFs and MSCs were mixed at a ratio of 50:50. Caco-2 cells alone were centrifuged at 200×g for 5 minutes. The resulting pellets were mixed with aqueous hyaluronic acid and aqueous gelatin at a concentration of 200 million cells/mL (IMFs and MSCs) and at a concentration of 125 million cells/mL (Caco-2) and loaded into the syringe.

### Example 9—Process for Forming a Tube with Four Layers

The prepared bio-ink of Example 8 was used in the following process. After 15 incubation at 4° C., syringes were loaded onto the printer for extrusion onto the rotating mandrel. Layers of cells were printed onto a 1 mm or 3 mm mandrel that had been dip-coated in or molded with NOVOGEL® 1.0. The resulting tube was encased in alginate and gelatin and cross-linked in aqueous calcium chloride for 1 minute. The mandrel and tube were transferred to media consisting of 50% SMGM-2 and 50% supplemented DMEM and were held static overnight at 30° C. in 5% CO₂. After 24 hours, the tubes were rotated at 10 rpm at 30° C. in 5% CO₂. After 48 hours, the tubes were rotated at 10 rpm at 37° C. and remained rotating on the mandrel. Tubes were removed from the mandrel and then fixed in 2% paraformaldehyde for histological processing.

### Example 10—Process for Forming a Single-Layered Tube

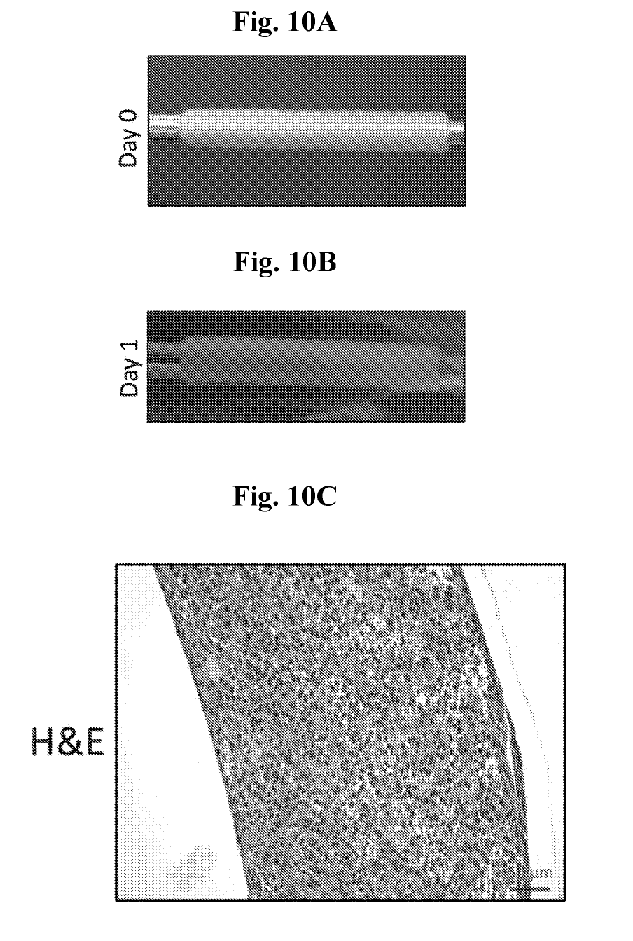

Based on the process of Example 8, a mixture of 50:50 IMF to human dermal fibroblasts (HDF) was prepared and mixed with aqueous hyaluronic acid and aqueous gelatin at a concentration of 200 million cells/mL (IMFs and HDFs). The resulting mixture was printed directly onto a mandrel to produce a tube as shown in FIG. 10A (after printing) and FIG. 10B (after one day).

FIG. 10C shows the histological results of the single-layered biological tube stained with hematoxylin and eosin (H&E).

### Example 11—Process for Forming a Double-Layered Tube

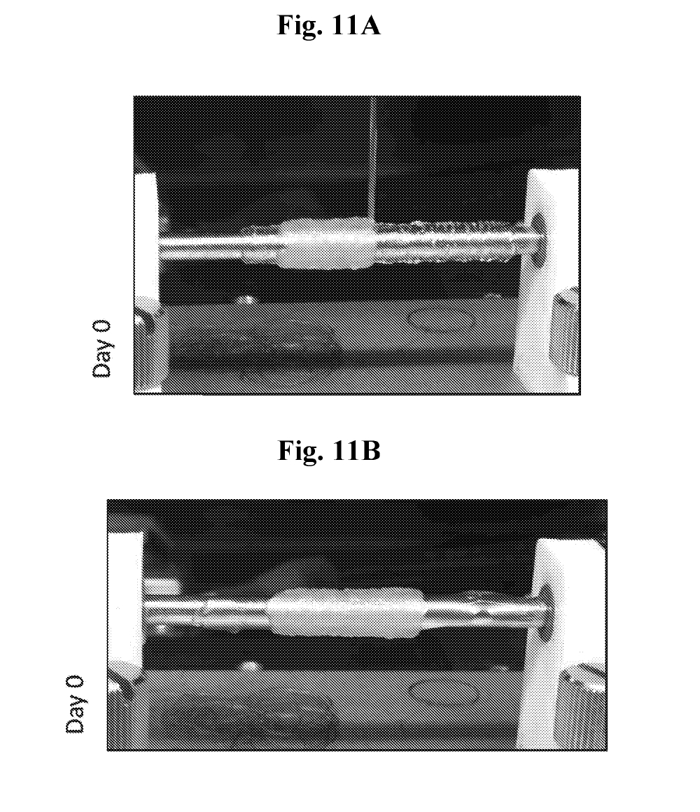

Based on the conditions used in Example 8, a mixture of 50:50 IMF to MSC was prepared and mixed with aqueous hyaluronic acid and aqueous gelatin at a concentration of 200 million cells/mL (IMFs and MSCs). FIG. 11A shows a double-layered tube with a first layer of alginate and gelatin that was cross-linked with aqueous calcium chloride and allowed to form a coating on the mandrel before printing with the IMF and MSC mixture to form a second layer. FIG. 11B shows a double-layered tube with a first layer formed by dip-coating with NOVOGEL® 1.0 and allowed to form a coating on the mandrel before printing with the IMF and MSC mixture to a form a second layer.

### Example 12—Process for Forming a Triple-Layered Tube

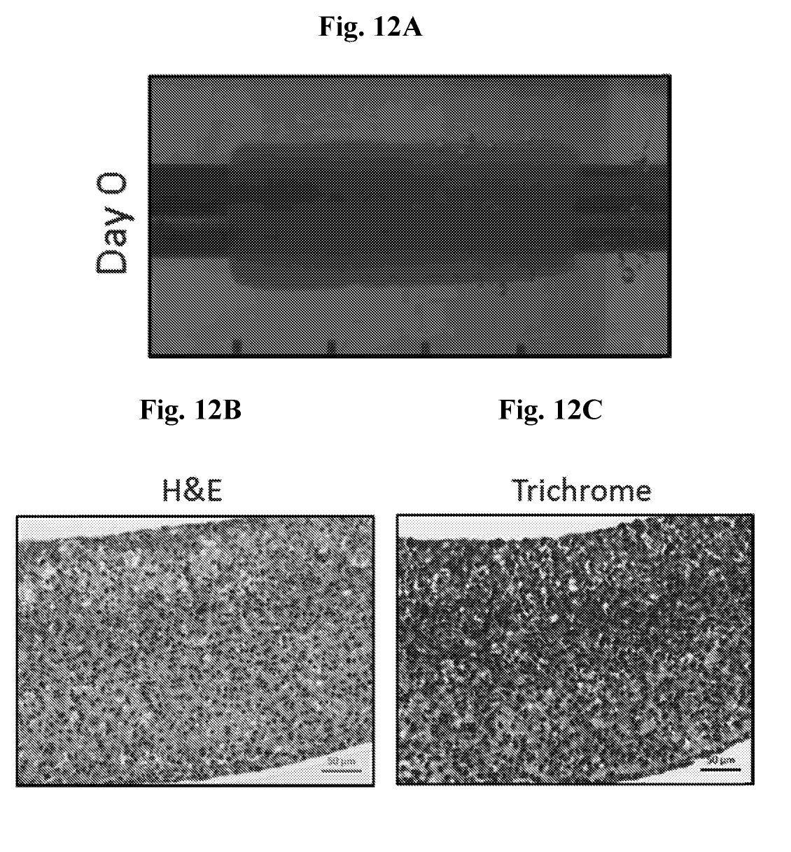

Based on the conditions used Example 8, a mixture of 50:50 IMF to MSC was prepared and mixed with hyaluronic acid and gelatin at a concentration of 200 million cells/mL (IMFs and MSCs). A triple layer tube was formed with a first layer by dip-coating with NOVOGEL® 1.0 and allowing to form a coating on the mandrel followed by printing with the IMF and MSC mixture to form a second layer which was encased in aqueous alginate and aqueous gelatin and cross-linked in aqueous calcium chloride for 1 minute. The resulting triple layered tube is shown after printing (FIG. 12A).

FIG. 12B shows the histological results of the triple-layered biological tube stained with H&E and FIG. 12C shows the histological results of staining with Masson's trichrome.

### Example 13—Process for Forming a Four-Layered Tube

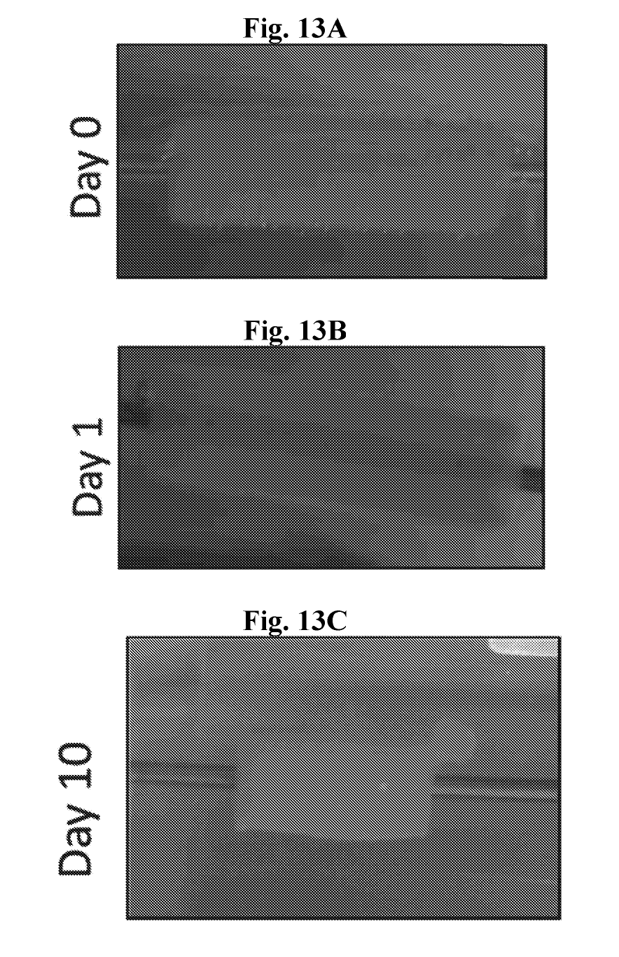

Based on the conditions used in Example 8, a mixture of 50:50 IMF to MSC was prepared and mixed with aqueous hyaluronic acid and aqueous gelatin at a concentration of 200 million cells/mL (IMFs and MSCs). A mixture of Caco-2 cells with aqueous hyaluronic acid and aqueous gelatin was prepared at a concentration of 125 million cells/mL. A first layer was formed by molding NOVOGEL® 1.0 onto the mandrel. Onto the first layer was printed a second layer comprising the mixture of Caco-2 cells using a 250 μm needle. Over the second layer was printed a third layer comprising the mixture of 50:50 IMF to MSC cells using a 500 μm needle. The resulting layers were encased in aqueous alginate and aqueous gelatin which was cross-linked in aqueous calcium chloride for 1 minute. After printing (day 0), the tubes were held statically at 30° C. overnight, then rotated at 10 rpm at 30° C. overnight, then moved to 37° C. with 10 rpm rotation for 3-10 days. The tubes are removed from the mandrel prior to fixation and processed for histology. The resulting four-layered tube is shown after printing (FIG. 13A), one day after printing (FIG. 13B), and 10 days after printing (FIG. 13C).

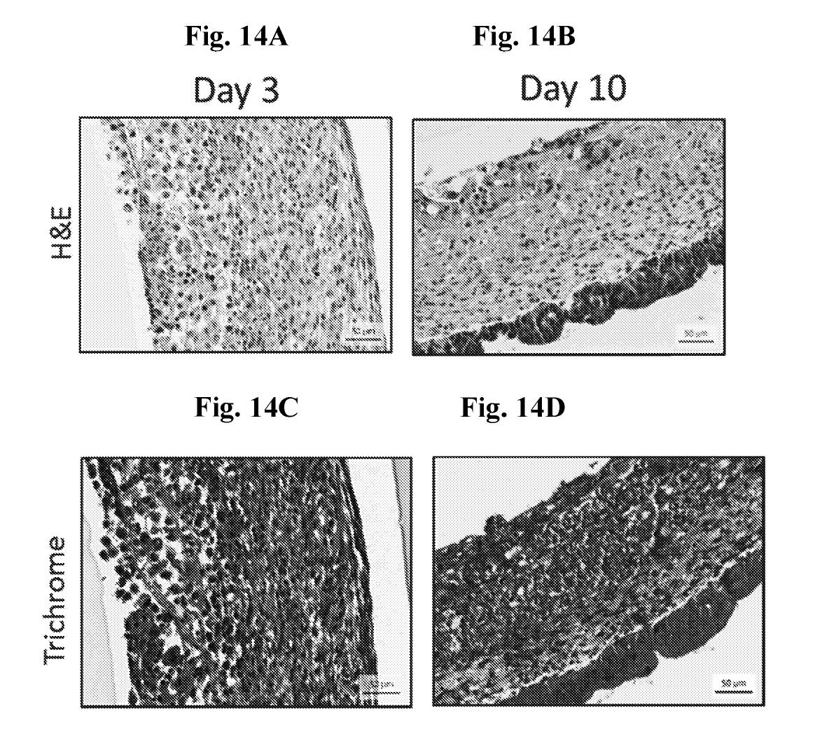

FIGS. 14A and 14B show the histological results of the four-layered biological tube stained with H&E after 3 days and 10 days, respectively, and FIGS. 14C and 14D shows the histological results of staining with Masson's trichrome after 3 days and 10 days, respectively.

### Example 14—Process for Forming a Four-Layered Tube (Layer 2 with Collagen and Gelatin)

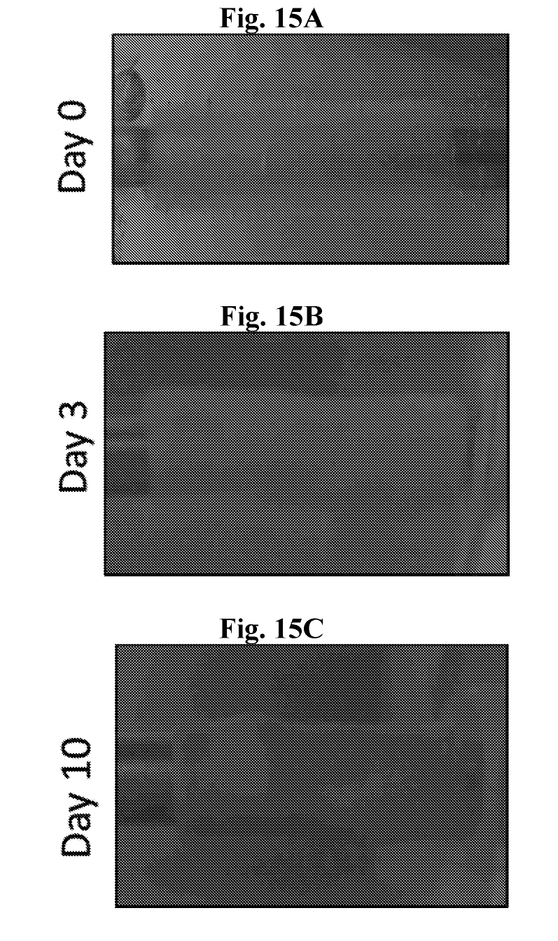

Based on the conditions used in Example 8, a mixture of 50:50 IMF to MSC was prepared and mixed with aqueous hyaluronic acid and aqueous gelatin at a concentration of 200 million cells/mL (IMFs and MSCs). A mixture of Caco-2 cells with aqueous collagen and aqueous gelatin was prepared at a concentration of 125 million cells/mL. A first layer was formed by molding NOVOGEL® 1.0 onto the mandrel. Onto the first layer was printed a second layer comprising the mixture of Caco-2 cells using a 250 μm needle. Over the second layer was printed a third layer comprising the mixture of 50:50 IMF to MSC cells using a 500 μm needle. The resulting layers were encased in aqueous alginate and aqueous gelatin which was cross-linked in aqueous calcium chloride for 1 minute. After printing (day 0), the tubes are held statically at 30° C. overnight, then rotated at 10 rpm at 30° C. overnight, then moved to 37° C. with 10 rpm rotation for 3-10 days. The tubes are removed from the mandrel prior to fixation and processing for histology. The resulting four layer tube is shown after printing (FIG. 15A), 3 days after printing (FIG. 15B) and 10 days after printing (FIG. 15C).

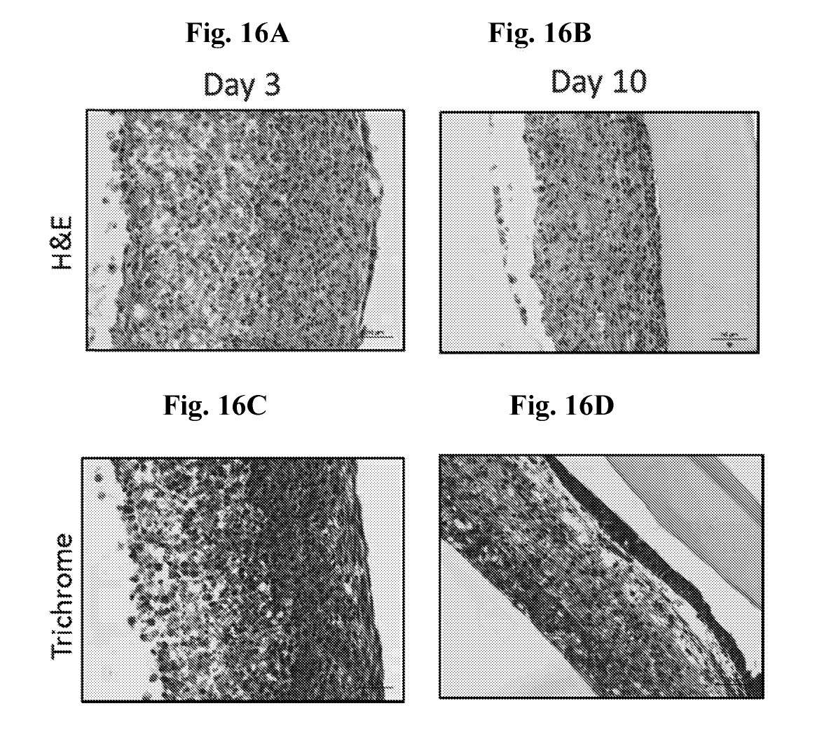

FIGS. 16A and 16B show the histological results of the four-layered biological tube stained with H&E after 3 days and 10 days, respectively, and FIGS. 16C and 16D show the histological results of staining with Masson's trichrome after 3 days and 10 days, respectively.

### Example 15—Process for Forming a Three-Layered Tube (Layer 2 with Caco-2, IMF, and MSC)

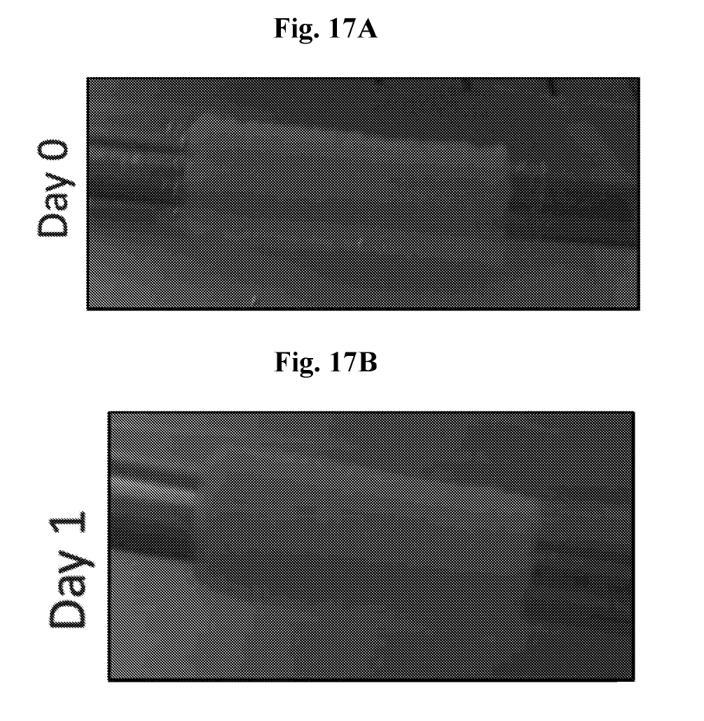

Based on the conditions used in Example 8, a mixture of IMF, MSC, and Caco-2 cells was prepared and mixed with aqueous hyaluronic acid and aqueous gelatin at a concentration of 200 million cells/mL (IMFs, MSCs, and Caco-2 cells). A first layer was formed by dip-coating the mandrel with NOVOGEL® 1.0. Onto the first layer was printed a second layer comprising the mixture of IMF, MSC, and Caco-2 cells using a 250 μm needle. The resulting layers were encased in aqueous alginate and aqueous gelatin which was cross-linked in aqueous calcium chloride for 1 minute. The resulting triple-layered tube is shown after printing (FIG. 17A) and one day after printing (FIG. 17B).

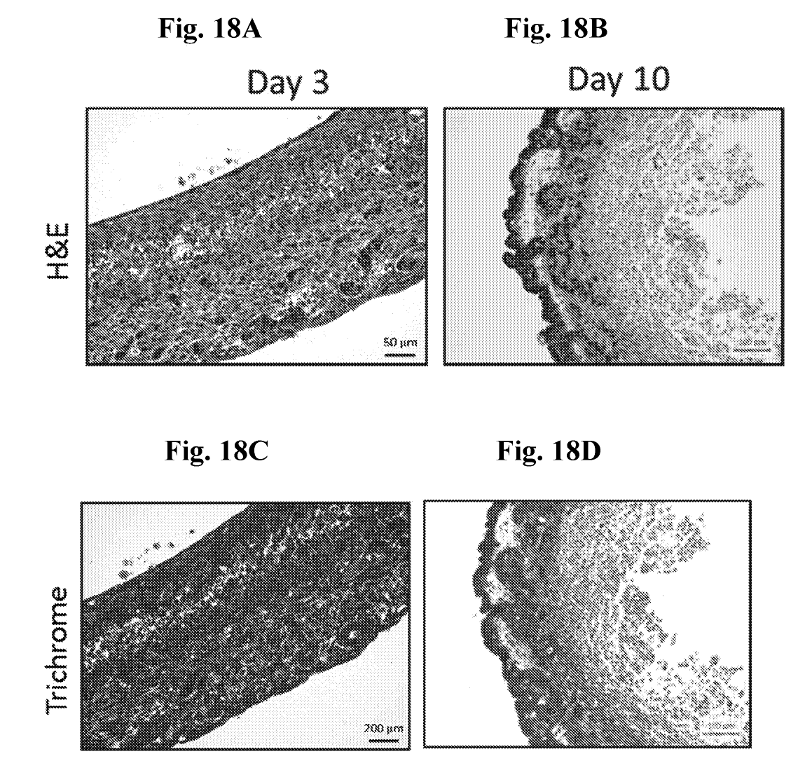

FIGS. 18A and 18B show the histological results of the four-layered biological tube stained with H&E after 3 days and 10 days, respectively, and FIGS. 18C and 18D show the histological results of staining with Masson's trichrome after 3 days and 10 days, respectively. As seen in FIGS. 18A and 18C, after 3 days, Caco-2 cells (CK19) are found randomly throughout the construct. As seen in FIGS. 18C and 18D, after ten days, Caco-2 cells have organized along the exterior of the tube.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patents, and patent applications mentioned in this specification are herein incorporated by reference to the same extent as if each individual publication, patent, or patent application was specifically and individually indicated to be incorporated by reference.

1. A bioprinter for fabricating biological tubes comprising:
(a) a printer head comprising: a reservoir containing bio-ink and a deposition orifice, the bio-ink is a solid or semi-solid composition comprising living cells;
(b) a calibration element for determining the position of the deposition orifice;
(c) an extrusion element for extruding the bio-ink through the deposition orifice by application of pressure;
(d) a rotating mandrel for receiving the extruded bio-ink, the rotating mandrel removable from the device;
(e) a motor for rotating the mandrel; and
(f) a programmable computer processor communicatively connected to the calibration element, the extrusion element, and the motor, the programmable computer processor for regulating motion of the printer head, regulating extrusion of the bio-ink, and regulating the rotation of the mandrel to fabricate a biological tube.

2. The bioprinter of claim 1, wherein the motor rotates at 10 rpm to 29 rpm; preferably, wherein the motor rotates at 17 rpm.

3. (canceled)

4. The bioprinter of claim 1, wherein the extrusion element extrudes the bio-ink at a volume of 0.10 μl/s to 0.50 Os.

5. (canceled)

6. The bioprinter of claim 1, wherein the mandrel is porous; preferably, wherein the mandrel is permeable to gas, liquid, or both gas and liquid.

7-10. (canceled)

11. The bioprinter of claim 1, wherein the mandrel is partially or completely covered with a removable sheath that is permeable to gas, liquid, or both gas and liquid.

12-18. (canceled)

19. The bioprinter of claim 1, wherein the bio-ink consists essentially of cells.

20-22. (canceled)

23. A method of fabricating a biological tube, the method comprising:
(a) depositing a continuous bio-ink, the bio-ink is a solid or semi-solid, the bio-ink comprising living cells onto a rotating biocompatible mandrel; and
(b) maturing the deposited bio-ink while on the mandrel in a cell culture media to allow the cells to cohere to form the biological tube.

24. The method of claim 23, further comprising:
(c) removing the biological tube from the mandrel; and
(d) maturing the biological tube in a cell culture media.

25-27. (canceled)

28. The method of claim 23, wherein the bioprinter continuously deposits the bio-ink at a rate of 0.020 mm/s to 0.050 mm/s.

29. (canceled)

30. The method of claim 23, wherein the bioprinter translates the position of the printer head across the length of the rotating mandrel during deposition of the bio-ink; preferably, wherein the bioprinter translates the position of the printer head at a rate of 0.03 mm/s to 0.25 mm/s.

31-33. (canceled)

34. The method of claim 23, wherein the bio-ink consists essentially of cells.

35-38. (canceled)

39. The method of claim 23, wherein the biological tube has a uniform wall thickness.

40-44. (canceled)

45. The method of claim 23, wherein the biological tube lacks at least one of innervation, lymphatic tissue, perfusable supporting vasculature, and red blood cells.

46-48. (canceled)

49. A system comprising a permeable, biocompatible tubular sheath and a continuous bio-ink comprising living cells wound around the sheath to form a biological tube.

50. The system of claim 49, further comprising a mandrel inside the tubular sheath.

51. (canceled)

52. A method of fabricating a multilayered biological tube, the method comprising:
(a) depositing a first continuous bio-ink, the first bio-ink a solid or semi-solid composition comprising living cells onto a rotating biocompatible mandrel;
(b) depositing a second continuous bio-ink, the second bio-ink a solid or semi-solid composition comprising living cells onto a rotating biocompatible mandrel;
(c) depositing a layer of cells onto the deposited second bio-ink; and
(d) maturing the deposited bio-inks and cells while on the mandrel in a cell culture media to allow the cells to cohere to form the biological tube.

53. The method of claim 52, further comprising:
(e) removing the biological tube from the mandrel; and
(f) maturing the biological tube in a cell culture media.

54-55. (canceled)

56. A method of fabricating a multilayered biological tube, the method comprising:
(a) depositing a layer of cells onto a rotating biocompatible mandrel;
(b) depositing a first continuous bio-ink, the first bio-ink a solid or semi-solid composition comprising living cells onto a rotating biocompatible mandrel;
(c) depositing a second continuous bio-ink, the second bio-ink a solid or semi-solid composition comprising living cells onto a rotating biocompatible mandrel; and
(d) maturing the deposited bio-ink and cells while on the mandrel in a cell culture media to allow the cells to cohere to form the biological tube.

57. The method of claim 56, further comprising:
(e) removing the biological tube from the mandrel; and
(f) maturing the biological tube in a cell culture media.

58-59. (canceled)

60. An engineered biological tube, the tube fabricated by a process comprising:
(a) depositing a continuous bio-ink, the bio-ink is a solid or semi-solid, the bio-ink comprising living cells onto a rotating biocompatible mandrel;
(b) maturing the deposited bio-ink while on the mandrel in a cell culture media to allow the cells to cohere to form the biological tube.

61. The tube of claim 60, further comprising:
(c) removing the biological tube from the mandrel; and
(d) maturing the biological tube in a cell culture media.

62-90. (canceled)
